(19) United States
(12) Patent Application Publication
(10) Pub. No.: US 2010/0190650 A1
(43) Pub. Date: Jul. 29, 2010

(54) TRANSCRIPTOMIC BIOMARKER OF MYOCARDITIS

(75) Inventors: Joshua M. Hare, Miami Beach, FL (US); Bettina Heidecker, Miami, FL (US)

Correspondence Address: NOVAK DRUCE + QUIGG LLP (WPB), 525 Okeechobee Blvd, 15th Floor, City Place Tower, West Palm Beach, FL, 33401, US

(73) Assignee: Univrsity of Miami, Miami, FL (US)

(21) Appl. No.: 12/609,194

(22) Filed: Oct. 30, 2009

Related U.S. Application Data
(63) Continuation-in-part of application No. PCT/US08/62290, filed on May 1, 2008.
(60) Provisional application No. 60/915,215, filed on May 1, 2007.

(51) Int. Cl. C40B 30/00; C07K 14/00; G01N 33/50; C12Q 1/68; G01N 33/53; C07H 21/00; C07K 16/00; C40B 40/06; C12N 15/74; C12N 5/071

(52) U.S. Cl. 506/7; 530/350; 436/86; 435/6; 435/7.1; 536/23.1; 530/387.1; 506/16; 435/320.1; 435/325

(57) ABSTRACT

Molecular signatures that function as very sensitive diagnostic biomarker for myocarditis, heart disease and disorders thereof, are identified.

### CROSS-REFERENCE TO RELATED APPLICATIONS

The present application claims the priority of International Application No.: PCT/US2008/62290, international filing date May 1, 2008, which in turn claims priority to filed U.S. provisional patent application No. 60/915,215 filed May 1, 2007 which are incorporated herein by reference in its entirety.

### STATEMENT REGARDING FEDERALLY SPONSORED RESEARCH

This invention was made with U.S. government support under grant numbers M400-217-2954 and RO-1 HL-65455 both awarded by the National Institutes of Health. The U.S. government may have certain rights in the invention.

### FIELD OF THE INVENTION

This invention relates to biomarkers of heart disease, myocarditis, novel drug therapeutic targets, compositions and methods of predicting, diagnosing and treating heart diseases and related disorders thereof. More specifically, the invention concerns methods and compositions based on unique molecular signatures associated with various aspects of cardiac diseases and disorders.

### BACKGROUND

The current approach to the treatment of patients with heart failure due to impaired cardiac function lacks individualization. This issue is of increasing importance as the number of classes of medicine for heart failure increase. Moreover there is growing appreciation that there may be utility to cause specific therapies. Accurate biomarkers are needed to refine diagnostic accuracy so as to enhance the application of personalized medicine in the field of heart failure.

There is a need in the art to provide early diagnosis and prognosis of heart disease. Myocarditis causes a significant minority of depressed heart function and thus causes heart failure and premature and unexpected sudden cardiac death. Myocarditis affects humans throughout life including children. The current diagnostic approach using histologic analysis of heart tissue obtained by biopsy lacks sensitivity and specificity.

### SUMMARY

This Summary is provided to present a summary of the invention to briefly indicate the nature and substance of the invention. It is submitted with the understanding that it will not be used to interpret or limit the scope or meaning of the claims.

Given high risk of developing serious cardiac complications from myocarditis and the availability of disease specific therapies, there is a need for better biomarkers, to adjust treatment appropriately and early enough.

Molecular signatures that function as very sensitive diagnostic biomarker for myocarditis, heart disease and disorders thereof, were identified.

In a preferred embodiment, a molecular composition comprises gene sequences: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE:5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, variants and/or gene products thereof.

In another preferred embodiment, detection of the gene sequences, complementary sequences, fragments, alleles, variants and gene products thereof, is diagnostic of myocarditis and myocardial disorders.

In another preferred embodiment, detection in a cell or patient of at least ten gene sequences, complementary sequences, fragments, alleles, variants and gene products thereof, is diagnostic of diagnostic of myocarditis, idiopathic cardiomyopathy, heart diseases and disorders thereof.

In another preferred embodiment, the gene sequences, complementary sequences, fragments, alleles, variants and gene products thereof, are over-expressed at levels by at least 1%, 2%, 5%, 10% in a cell or patient as compared to levels in a normal cell or normal subject.

A biomarker (TBB-I) for the diagnosis of myocarditis comprises: nucleic acid sequences/biomolecules comprising: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE: 5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, variants and gene products thereof, complementary sequences, fragments, alleles, variants and/or gene products thereof.

In an alternative embodiment, detection of at least ten biomolecules is diagnostic of myocarditis.

In another preferred embodiment, an antibody or aptamer specific for each gene sequence comprising nucleic acid sequences/biomolecules comprising: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE: 5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, variants and gene products thereof, complementary sequences, fragments, alleles, variants and/or gene products thereof.

In another preferred embodiment, a biochip comprises nucleic acid sequences: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE:5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, variants and gene products thereof, complementary sequences, fragments, alleles, variants and/or gene products thereof.

In another preferred embodiment, the biochip comprises at least ten nucleic sequences, complementary sequences, fragments, alleles, variants and gene products thereof.

In one preferred embodiment, a transcriptomic biomarker (TBB-II) comprises gene sequences: 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), 43934_at (G protein-coupled receptor 137), complementary sequences, fragments, alleles, variants and/or gene products thereof.

In another preferred embodiment, the detection of the gene sequences, complementary sequences, fragments, alleles, variants and gene products thereof, is diagnostic of myocarditis and myocardial disorders, cardiac and cardiovascular diseases and disorders, such as for example, Coronary Heart Disease, angina, Acute Coronary Syndrome, Aortic Aneurysm and Dissection, arrhythmias, Cardiomyopathy, Congenital Heart Disease, congestive heart failure or chronic heart failure, pericarditis, and the like.

In another alternative embodiment, detection in a cell or patient of at least ten gene sequences, complementary sequences, fragments, alleles, variants and gene products thereof, is diagnostic of diagnostic of myocarditis, idiopathic cardiomyopathy, heart diseases and disorders thereof.

In another embodiment, the gene sequences, complementary sequences, fragments, alleles, variants and gene products thereof, are modulated at levels by at least about 1%, 5%, 10%, 100%, 200% or more in a cell or patient as compared to levels in a control, normal samples or normal subject.

In another preferred embodiment, a biochip comprises nucleic acid sequences: 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), 43934_at (G protein-coupled receptor 137), complementary sequences, fragments, alleles, variants and/or gene products thereof.

In yet another embodiment, an antibody or aptamer specific for each gene sequence comprising nucleic acid sequences/biomolecules comprising: 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), 43934_at (G protein-coupled receptor 137), complementary sequences, fragments, alleles, variants and/or gene products thereof.

In another embodiment, a biomarker (TBB-III) comprises nucleic acid sequences/biomolecules comprising: 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE:5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 243766_s_at (TEA domain family member 2), and 244042_x_at complementary sequences, fragments, alleles, variants and/or gene products thereof.

In another preferred embodiment, a method of diagnosing myocarditis, comprises identifying in a biological sample from a patient a molecular signature of nucleic acid sequences/biomolecules comprising transcriptomic based biomarker-I (TBB-I) comprising: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE:5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, variants and gene products thereof; assessing the probability of identification of each component gene in each sample; assigning each to a class; and, differentiating between idiopathic cardiomyopathy and myocarditis.

In another preferred embodiment, a method of diagnosing heart disease or myocarditis comprises identifying in a biological sample from a patient a molecular signature of nucleic acid sequences/biomolecules comprising transcriptomic based biomarker-II (TBB-II) comprising: 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), 43934_at (G protein-coupled receptor 137), complementary sequences, fragments, alleles, variants and gene products thereof; assessing the probability of identification of each component gene in each sample; assigning each to a class; and, diagnosing heart disease or myocarditis.

In another preferred embodiment, a method of diagnosing heart disease or myocarditis comprises identifying in a biological sample from a patient a molecular signature of nucleic acid sequences/biomolecules comprising transcriptomic based biomarker-III (TBB-III) comprising: 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE:5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 243766_s_at (TEA domain family member 2), and 244042_x_at, complementary sequences, fragments, alleles, variants and gene products thereof; assessing the probability of identification of each component gene in each sample; assigning each to a class; and, diagnosing heart disease or myocarditis.

In another preferred embodiment a kit comprises a transcriptomic based biomarker-I (TBB-I): 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE:5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 43766_s_at (TEA domain family member 2), and 244042_x_at complementary sequences, fragments, alleles, variants and/or gene products thereof.

In another preferred embodiment, a kit comprises a transcriptomic based biomarker-II 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), 43934_at (G protein-coupled receptor 137), complementary sequences, fragments, alleles, variants and/or gene products thereof.

In another preferred embodiment, a kit comprises a transcriptomic based biomarker-III (TBB-III): 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE:5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 243766_s_at (TEA domain family member 2), and 244042_x_at complementary sequences, fragments, alleles, variants and/or gene products thereof.

In another embodiment, a cell expresses one or more biomolecules comprising the biomarker TBB-I.

In another embodiment, a cell expresses one or more biomolecules comprising the biomarker TBB-II.

In another embodiment, a cell expresses one or more biomolecules comprising the biomarker TBB-III

In another preferred embodiment, a vector encodes one or more biomolecules comprising TBB-I.

In another preferred embodiment, a vector encodes one or more biomolecules comprising TBB-II.

In another preferred embodiment, a vector encodes one or more biomolecules comprising TBB-III.

In another embodiment, a cell expresses one or more biomolecules comprising the biomarker TBB-I, TBB-II, TBB-II or combinations thereof.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 shows a SAM plot of samples from patients with idiopathic cardiomyopathy versus patients with myocarditis: Red color denotes genes that were significantly upregulated in patients with myocarditis, green color denotes genes that were significantly downregulated. The outer blue lines represent the positive and negative cutoff points that were chosen with the delta value. 134 genes were significantly different between the two groups, when a delta value of 1.41 was chosen (FDR=0.49). We reduced this subset of genes to 122 candidate genes with a q-value of 0% that we used for hierarchical clustering and PAM analysis.

FIG. 2 shows a heatmap created by hierarchical clustering using 122 candidate genes: Samples from patients with myocarditis are labeled “Myo-”, samples from patients with idiopathic cardiomyopathy are labeled either with “GP-” or “BP-”. Each column represents a sample and each line corresponds to a gene, for which the IDs from Affymetrix are listed on the right side. For further details about the gene annotations see Table 1. A red color means low expression of the gene, whereas a blue color demonstrates high gene expression levels. Six samples were grouped wrong.

FIG. 3 is a graph showing the misclassification error of the training set. The classifier was trained on 8 samples from patients with myocarditis and 25 samples from patients with idiopathic cardiomyopathy. After increasing the threshold to 3.2 and reducing the genes of the classifier to less than 22, the misclassification error increased dramatically.

FIG. 4 is a graph showing results from the “39 genes classifier” for myocarditis: This graph visualizes the calculated probabilities for each class after the “39 genes molecular signature” was applied with a threshold of 2.6. The probability can be read from the y-axis. Group 1 represents the samples from patients with idiopathic cardiomyopathy, group 2 represents the samples from patients with myocarditis.

FIG. 5 shows the nearest shrunken centroid of the “39 genes classifier”: The centroids were calculated in PAM from the average expression for each gene in each class divided by the within-class standard deviation for that gene. The nearest shrunken centroid classification “shrinks” each of the class centroids toward the overall centroid for all classes by the threshold. Nearest centroid classification takes the gene expression profile of a new sample, and compares it to each of these class centroids. The class whose centroid that it is closest to, in squared distance, is the predicted class for that new sample. Each line in the graph represents a gene. The red centroid characterizes group 1 (idiopathic cardiomyopathy), the green centroid characterizes group 2 (myocarditis). Upregulation is illustrated as a vector to the right, downregulation as a vector to the left on the graph.

FIG. 6 shows a heatmap of the “39 genes transcriptomic biomarker”: This heatmap was created by the same unsupervised clustering method as FIG. 2.

FIG. 7 shows the Principal Components Analysis (PCA) and various Cluster Algorithms in samples of myocarditis vs. other types of cardiomyopathy: To illustrate the contribution of each of the 122 genes (FC >1.2; q <0.1%) to every phenotype, we performed PCA (n=61). We used correlation matrix with genes as variables. Less significant genes are denoted with vectors close to the center and correspond to genes that were excluded using PAM analysis. Myocarditis samples were labeled with “M”, samples from patients with other forms of cardiomyopathy were labeled with “O”. Genes that were overexpressed are labeled with serial numbers and are clustered with the corresponding class. All myocarditis samples, except two, were clearly grouped together. We further tested the robustness of the reduced set of 39 genes with 3 different types of clustering algorithms (Ward's method, complete linkage and average linkage). Myocarditis samples are highlighted in red—incorrectly classified samples are encircled in blue. All methods achieved the same accuracy as PAM (n=33, 97% accuracy).

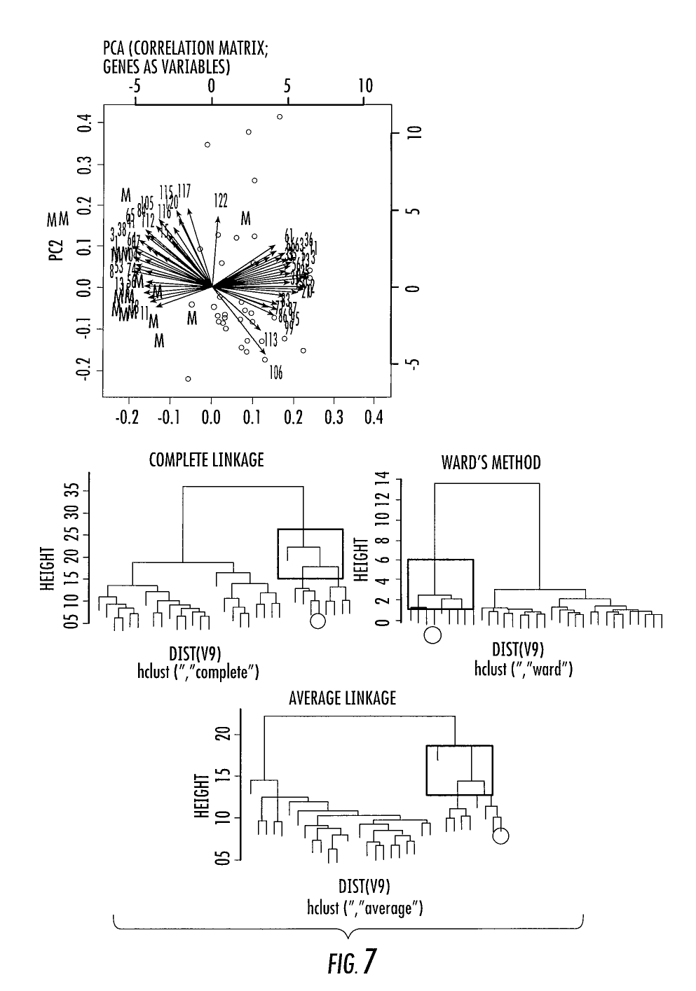

### DETAILED DESCRIPTION

The invention comprises molecular signatures that function as a very sensitive diagnostic biomarker for heart failure, heart diseases, myocarditis, and other heart disorders. Myocarditis is a common disease that is estimated to cause up to 30% of dilated cardiomyopathy, even in patients initially asymptomatic. Myocarditis can also present as sudden cardiac death and affects individuals of all ages. In childhood, myocarditis causes a greater percentage of heart failure than in adulthood. The fact that the majority of viral induced cases pass in a clinically unapparent course, points out the significance of finding more reliable biomarkers than standard diagnostic tools which are currently available, e.g. ECG, cardiac enzymes and immunohistochemistry.

Current standard diagnostic tools for myocarditis (ECG, cardiac enzymes, immunohistochemistry) are not always reliable enough and many patients undergo clinically unapparent courses without getting treated. Especially in pediatrics it is crucial to detect myocarditis in an early stage as a fatal course has been observed many times in children.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, a “molecular signature” or “signature” or “biomarker” or “transcriptomic based biomarker” are used interchangeably herein and refers to all the biomolecules identified in Tables 1, 2, and 4. Thus, Table 1 comprising the biomolecules listed therein, represents one biomarker or molecular signature; Table 2 comprising the biomolecules listed therein, represents another one biomarker or molecular signature; and so forth. As more biomolecules are discovered, each newly identified biomolecules can be assigned to any one or more biomarker or molecular signature. Each biomolecule can also be removed, reassigned or reallocated to a molecular signature. Thus, in some embodiments the molecular signature comprises at least ten biomolecules. The ten biomolecules are selected from the genes identified herein, or from newly identified biomolecules. The biomarker identified by Table 1 comprises the 38 upregulated genes or markers and is termed TBB-I for brevity. The biomarker comprising the biomolecules or markers in Table 2 are termed TBB-II for brevity. The biomarker comprising the biomolecules or markers in Table 4 is termed TBB-III for brevity. Any one of TBB-I, TBB-II and TBB-III or combinations thereof can be used in the diagnosis of myocarditis. Any one of TBB-I, TBB-II and TBB-III or combinations thereof can be used in the diagnosis of myocarditis and idiopathic cardiomyopathy and differentiating between the two conditions.

The terms “biomolecule” or “markers” are used interchangeably herein and refer to DNA, RNA (including mRNA, rRNA, tRNA and tmRNA), nucleotides, nucleosides, analogs, polynucleotides, peptides and any combinations thereof.

A base “position” as used herein refers to the location of a given base or nucleotide residue within a nucleic acid.

As used herein, the term “array” refers to an ordered spatial arrangement, particularly an arrangement of immobilized biomolecules.

As used herein, the term “addressable array” refers to an array wherein the individual elements have precisely defined x and y coordinates, so that a given element at a particular position in the array can be identified.

As used herein, the terms “probe” and “biomolecular probe” refer to a biomolecule used to detect a complementary biomolecule. Examples include antigens that detect antibodies, oligonucleotides that detect complimentary oligonucleotides, and ligands that detect receptors. Such probes are preferably immobilized on a microelectrode comprising a substrate.

As used herein, the terms “bioarray,” “biochip” and “biochip array” refer to an ordered spatial arrangement of immobilized biomolecules on a microelectrode arrayed on a solid supporting substrate. Preferred probe molecules include aptamers, nucleic acids, oligonucleotides, peptides, ligands, antibodies and antigens; peptides and proteins are the most preferred probe species. Biochips, as used in the art, encompass substrates containing arrays or microarrays, preferably ordered arrays and most preferably ordered, addressable arrays, of biological molecules that comprise one member of a biological binding pair. Typically, such arrays are oligonucleotide arrays comprising a nucleotide sequence that is complementary to at least one sequence that may be or is expected to be present in a biological sample. Alternatively, and preferably, proteins, peptides or other small molecules can be arrayed in such biochips for performing, inter alia, immunological analyses (wherein the arrayed molecules are antigens) or assaying biological receptors (wherein the arrayed molecules are ligands, agonists or antagonists of said receptors).

Expression or amount of a gene, biomolecule, or marker in a first sample is at a level “greater than” the level in a second sample if the expression level/amount of the gene or biomarker in the first sample is at least about 1 time, 1.2 times, 1.5 times, 1.75 times; 2 times, 3 times, 4 times, 5 times, 6 times, 7 times, 8 times, 9 times, 10 times, 20 times, 30 times, the expression level/amount of the gene or biomarker in the second sample or a normal sample. Expression levels/amounts can be determined based on any suitable criterion known in the art, including but not limited to mRNA, cDNA, proteins, protein fragments and/or gene copy. Expression levels/amounts can be determined qualitatively and/or quantitatively. The term “expression” or “amount” also refers to a down regulation of a gene, biomolecule, or marker so that these molecules or markers in a first sample are at a level “less than” the level in a second sample if the expression level/amount of the gene or biomarker in the first sample is at least about 1 time, 1.2 times, 1.5 times, 1.75 times, 2 times, 3 times, 4 times, 5 times, 6 times, 7 times, 8 times, 9 times, 10 times, 20 times, 30 times, less than the expression level/amount of the gene or marker in the second sample or a normal sample.

By the term “modulate,” it is meant that any of the mentioned activities, are, e.g., increased, enhanced, increased, agonized (acts as an agonist), promoted, decreased, reduced, suppressed blocked, or antagonized (acts as an antagonist). Modulation can increase activity more than 1-fold, 2-fold, 3-fold, 5-fold, 10-fold, 100-fold, etc., over baseline values. Modulation can also decrease its activity below baseline values.

An “allele” or “variant” is an alternative form of a gene. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence. The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) or single base mutations in which the polynucleotide sequence varies by one base. The presence of SNPs may be indicative of, for example, a certain population with a propensity for a disease state, that is susceptibility versus resistance.

Derivative polynucleotides include nucleic acids subjected to chemical modification, for example, replacement of hydrogen by an alkyl, acyl, or amino group. Derivatives, e.g., derivative oligonucleotides, may comprise non-naturally-occurring portions, such as altered sugar moieties or inter-sugar linkages. Exemplary among these are phosphorothioate and other sulfur containing species which are known in the art. Derivative nucleic acids may also contain labels, including radionucleotides, enzymes, fluorescent agents, chemiluminescent agents, chromogenic agents, substrates, cofactors, inhibitors, magnetic particles, and the like.

A “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

The term, “complementary” means that two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. Normally, the complementary sequence of the oligonucleotide has at least 80% or 90%, preferably 95%, most preferably 100%, complementarity to a defined sequence. Preferably, alleles or variants thereof can be identified. A BLAST program also can be employed to assess such sequence identity.

The term “complementary sequence” as it refers to a polynucleotide sequence, relates to the base sequence in another nucleic acid molecule by the base-pairing rules. More particularly, the term or like term refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99% to about 100%. Complementary polynucleotide sequences can be identified by a variety of approaches including use of well-known computer algorithms and software, for example the BLAST program.

As used herein, the term “aptamer” or “selected nucleic acid binding species” is a nucleic acid macromolecule that shall include non-modified or chemically modified RNA or DNA, and binds tightly (as measured by Kₐ or K_{d}) to a specific molecular target. Like all nucleic acids, a particular aptamer may be described by a linear sequence of nucleotides (A, U, T, C and G), typically 15-40 nucleotides long. In solution, the chain of nucleotides forms intramolecular interactions that fold the molecule into a complex three-dimensional shape. The shape of the aptamer allows it to bind tightly against the surface of its target molecule. The method of selection may be by, but is not limited to, affinity chromatography and the method of amplification by reverse transcription (RT) or polymerase chain reaction (PCR).

As used herein, the term “signaling aptamer” shall include aptamers with reporter molecules, preferably a fluorescent dye, appended to a nucleotide in such a way that upon conformational changes resulting from the aptamer's interaction with a ligand, the reporter molecules yields a differential signal, preferably a change in fluorescence intensity.

As used herein, the term “fragment or segment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid or peptide fragment, more preferably at least about three contiguous nucleic acid bases or amino acid positions in common, most preferably at least about ten contiguous nucleic acid bases amino acid positions in common.

“Biological samples” include solid and body fluid samples. Preferably, the sample is obtained from heart. However, the biological samples used in the present invention can include cells, protein or membrane extracts of cells, blood or biological fluids such as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils and thymus. Examples of “body fluid samples” include, but are not limited to blood, serum, semen, prostate fluid, seminal fluid, urine, saliva, sputum, mucus, bone marrow, lymph, and tears.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The term “diagnosis”, as used in this specification refers to predict the type of disease or condition from a set of marker values and/or patient symptoms. This is in contrast to disease prediction, which is to predict the occurrence of disease before it occurs, and the term “prognosis”, which is to predict disease progression at a future point in time from one or more indicator value(s) at a previous point in time.

The term “correlating,” as used in this specification refers to a process in which a set of examples of clinical inputs from subjects, such as marker levels, and their corresponding outputs, such as whether a subject suffered from heart failure, are related to each other. This relationship can be determined by comparing such examples to examples from a control and/or disease-free population at a later point in time, and selecting those indicators which can differentiate between the two disease states as a function of time alone or in combination at a certain probability level. The selection process is described herein. The selected markers, each at a certain level range which might be a simple threshold, are said to be correlative or associative with one of the disease states. Said correlated markers can be then be used for disease detection, diagnosis, prognosis and/or treatment outcome. Preferred methods of correlating markers is by performing marker selection as described in detail in the examples section which follows. Methods can include a feature selection algorithm, statistics and classification by mapping functions described herein. A preferred probability level is a 3% chance, 5% chance, a 7% chance, a 10% chance, a 15% chance, a 20% chance, a 25% chance, a 30% chance, a 35% chance, a 40% chance, a 45% chance, a 50% chance, a 55% chance, a 60% chance, a 65% chance, a 70% chance, a 75% chance, a 80% chance, a 85% chance, a 90% chance, a 95% chance, and a 100% chance. Each of these values of probability is plus or minus 2% or less.

The terms “detecting”, “detect”, “identifying”, “quantifying” includes assaying, quantitating, imaging or otherwise establishing the presence or absence of the transcriptomic biomarker, or combinations of biomolecules comprising the biomarker, and the like, or assaying for, imaging, ascertaining, establishing, or otherwise determining the prognosis and/or diagnosis of heart failure.

Transcriptomic Biomarker/Molecular Signatures

In a preferred embodiment, a biomarker (TBB-I) comprises nucleic acid sequences/biomolecules comprising: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin/1/calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE: 5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, derivatives, variants and/or gene products thereof.

In another preferred embodiment, the biomolecules comprising: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE:5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2) are upregulated in patients with myocarditis as compared to normal subjects. In some embodiments at least ten biomolecules are modulated or upregulated as compared to controls.

In another preferred embodiment, a transcriptomic biomarker (TBB-II) comprises biomolecules comprising: 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at (kinase suppressor of ras 1), 214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), 43934_at (G protein-coupled receptor 137), complementary sequences, fragments, alleles, derivatives, variants and/or gene products thereof.

In another preferred embodiment, the biomolecules comprising the transcriptomic biomarker (TBB-II) complementary sequences, fragments, alleles, derivatives, variants and/or gene products thereof, are modulated in a patient suffering from myocarditis as compared to a normal subject.

In another preferred embodiment, a biomarker (TBB-III) comprises nucleic acid sequences/biomolecules comprising: 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE:5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 243766_s_at (TEA domain family member 2), and 244042_x_at complementary sequences, fragments, alleles, derivatives, variants and/or gene products thereof.

In another preferred embodiment, the detection of any one of the biomarkers, TBB-I, TBB-II and TBB-III or combinations thereof, are diagnostic of myocarditis, idiopathic cardiomyopathy, heart diseases and disorders thereof. In one embodiment, the biomolecules are modulate with respect to each other and produce a signature or expression profile for diagnosis and prognosis of specific diseases. For example, arrythmogenic right ventricular dysplasia, stress induced cardiomyopathy, myocarditis, systemic lupus erythematosus, sarcoidosis, peripartum cardiomyopathy, idiopathic dilated cardiomyopathy, cardiovascular diseases or disorders, etc.

In another preferred embodiment, the detection of at least ten biomolecules in TBB-I is diagnostic of myocarditis, idiopathic cardiomyopathy, heart diseases and disorders thereof.

In another preferred embodiment, the detection of at least ten molecules in TBB-II is diagnostic of myocarditis, idiopathic cardiomyopathy, arrythmogenic right ventricular dysplasia, stress induced cardiomyopathy, systemic lupus erythematosus, sarcoidosis, peripartum cardiomyopathy, idiopathic dilated cardiomyopathy, heart diseases and disorders thereof. Since the biomolecules in TBB-II are downregulated in myocarditis, when the term “detection” is used with respect to the biomolecules of TBB-II, will refer to the downregulation of the biomolecules as compared to the expression in normal or healthy cells or subjects. Detection will also refer to the up-regulation of any biomolecule in the signature, or expression of each biomolecule with respect to each other and controls.

In another preferred embodiment, the detection of at least ten molecules in TBB-III is diagnostic of myocarditis, idiopathic cardiomyopathy, arrythmogenic right ventricular dysplasia, stress induced cardiomyopathy, systemic lupus erythematosus, sarcoidosis, peripartum cardiomyopathy, idiopathic dilated cardiomyopathy, heart diseases and disorders thereof.

In another preferred embodiment, the detection in a cell or patient of the biomolecules, complementary sequences, fragments, alleles, variants and gene products thereof, is diagnostic of myocarditis, idiopathic cardiomyopathy, arrythmogenic right ventricular dysplasia, stress induced cardiomyopathy, systemic lupus erythematosus, sarcoidosis, peripartum cardiomyopathy, idiopathic dilated cardiomyopathy, heart diseases and disorders thereof. Preferably, the biomolecule sequences, complementary sequences, fragments, alleles, variants and gene products thereof, are modulated at levels by at least between 1%, 2%, 5%, 10% in a cell or patient as compared to levels in a normal cell or normal subject; more preferably, the gene biomarker sequences, complementary sequences, fragments, alleles, variants and gene products thereof, are modulated by about 50% in a biological sample, control or a patient as compared to levels in a normal biological sample, control or normal subject; more preferably, the gene biomarker sequences, complementary sequences, fragments, alleles, variants and gene products thereof, are modulated by about 75% in a biological sample or a patient as compared to levels in a control, normal biological sample, control or normal subject. The term “modulated” also refers to an increase or decrease in level, concentration, amount etc, as compared to a normal biological sample, control or normal healthy subject.

In another preferred embodiment, a biochip comprises a molecular signature comprising TBB-I, TBB-II, or TBB-III, complementary sequences, fragments, alleles, variants and gene products thereof.

In another preferred embodiment, a biochip comprises at least ten biomolecules selected from the biomolecules comprising TBB-I, TBB-II, or TBB-III, complementary sequences, fragments, alleles, variants and gene products thereof. Any ten or more can be selected from either one of TBB-I, TBB-II, or TBB-III biomarkers or selected form combinations thereof. There is nothing to preclude adding any newly identified biomarkers or any other known biomarkers or biomolecules thereof.

In another preferred embodiment, a method of differentiating between idiopathic cardiomyopathy and myocarditis, comprises: identifying in a biological sample from a patient a molecular signature comprising a transcriptomic based biomarker TBB-I, TBB-II and TBB-III.

In another preferred embodiment, a method of differentiating between idiopathic cardiomyopathy and myocarditis, comprises identifying in a biological sample from a patient a molecular signature comprising the biomolecules of transcriptomic based biomarker (TBB) complementary sequences, fragments, alleles, derivatives, variants, gene products or combinations thereof assessing the probability of identification of each component gene in each sample; and assigning each to a class.

In another preferred embodiment, the biomolecules are selected from TBB-I, TBB-II, and TBB-III, or combinations thereof.

In a preferred embodiment, phenotype specificity is identified by creating a classifier in a training set comprising about 66% of data obtained, with subsequent validation in a test set comprising about 33% of data obtained and defining a phenotype specific nearest shrunken centroid for classification.

In another preferred embodiment, a method of diagnosing heart disease or myocarditis comprises identifying in a biological sample from a patient a molecular signature comprising a transcriptomic based biomarker (TBB), TBB-I, TBB-II and TBB-III.

In another preferred embodiment, a method of diagnosing heart disease or myocarditis comprises detection of at least ten biomolecules, complementary sequences, fragments, alleles, variants and gene products thereof in a sample; assessing the probability of identification of each component gene in each sample; assigning each to a class; and, diagnosing heart disease arrythmogenic right ventricular dysplasia, stress induced cardiomyopathy, systemic lupus erythematosus, sarcoidosis, peripartum cardiomyopathy, idiopathic dilated cardiomyopathy, or myocarditis.

Alternative Methods and Materials for Identifying Molecular Signatures or Transcriptomic Biomarkers

Detection of Nucleic Acids and Proteins as Markers: In preferred embodiments, each biomarker is detected on chip based methods such as those described in detail in the examples which follow. In order to provide accurate diagnosis of cardiac disorders and diseases, for example, heart failure, myocarditis, idiopathic cardiomyopathy and the like. Other methods are also known in the art and one or more methods can be utilized.

The methods and assays disclosed herein are directed to the examination of expression of transcriptomic biomarkers in a mammalian tissue or cell sample, wherein the determination of that expression of one or more such transcriptomic biomarkers is predictive of prognostic outcome or diagnostic of cardiac and cardiovascular diseases and disorders, such as for example, myocarditis, Coronary Heart Disease, angina, Acute Coronary Syndrome, Aortic Aneurysm and Dissection, arrhythmias, Cardiomyopathy, Congenital Heart Disease, congestive heart failure or chronic heart failure, pericarditis, arrythmogenic right ventricular dysplasia, stress induced cardiomyopathy, systemic lupus erythematosus, sarcoidosis, peripartum cardiomyopathy, idiopathic dilated cardiomyopathy, and the like. The Molecular signatures or Transcriptomic biomarker comprise the biomolecules identified in Tables 1, 2, and 4. When making a diagnosis it is desirable to detect at least 10 or more biomolecules. Any one of TBB-I (Table 1), TBB-II (Table 2), TBB-III (Table 4) or combinations thereof can be used in the diagnosis of myocarditis. Any one of TBB-I, TBB-II and TBB-III or combinations thereof can be used in the diagnosis of myocarditis and idiopathic cardiomyopathy and differentiating between the two conditions.

Preferred embodiments in the identification of biomolecules, analytical methods etc, are described in detail in the Examples which follow.

Microarrays: In general, using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes that have potential to be expressed in certain disease states may be arrayed on a solid support. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment. (see, e.g., WO 01/75166 published Oct. 11, 2001; (See, for example, U.S. Pat. No. 5,700,637, U.S. Pat. No. 5,445,934, and U.S. Pat. No. 5,807,522, Lockart, Nature Biotechnology, 14:1675-1680 (1996); Cheung, V. G. et al., Nature Genetics 21 (Suppl): 15-19 (1999) for a discussion of array fabrication). DNA microarrays are miniature arrays containing gene fragments that are either synthesized directly onto or spotted onto glass or other substrates. Thousands of genes are usually represented in a single array. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. Currently two main types of DNA microarrays are being used: oligonucleotide (usually 25 to 70 mers) arrays and gene expression arrays containing PCR products prepared from cDNAs. In forming an array, oligonucleotides can be either prefabricated and spotted to the surface or directly synthesized on to the surface (in situ). The Affymetrix GENECHIP™ system is a commercially available microarray system which comprises arrays fabricated by direct synthesis of oligonucleotides on a glass surface.

Probe/Gene Arrays: Oligonucleotides, usually 25 mers, are directly synthesized onto a glass wafer by a combination of semiconductor-based photolithography and solid phase chemical synthesis technologies. Each array contains up to 400,000 different oligonucleotides and each oligonucleotide is present in millions of copies. Since oligonucleotide probes are synthesized in known locations on the array, the hybridization patterns and signal intensities can be interpreted in terms of gene identity and relative expression levels by the Affymetrix Microarray Suite software. Each gene is represented on the array by a series of different oligonucleotide probes. Each probe pair consists of a perfect match oligonucleotide and a mismatch oligonucleotide. The perfect match probe has a sequence exactly complimentary to the particular gene and thus measures the expression of the gene. The mismatch probe differs from the perfect match probe by a single base substitution at the center base position, disturbing the binding of the target gene transcript. This helps to determine the background and nonspecific hybridization that contributes to the signal measured for the perfect match oligonucleotide. The Microarray Suite software subtracts the hybridization intensities of the mismatch probes from those of the perfect match probes to determine the absolute or specific intensity value for each probe set. Probes are chosen based on current information from GenBank and other nucleotide repositories. The sequences are believed to recognize unique regions of the 3′ end of the gene. A GeneChip Hybridization Oven (“rotisserie” oven) is used to carry out the hybridization of up to 64 arrays at one time. The fluidics station performs washing and staining of the probe arrays. It is completely automated and contains four modules, with each module holding one probe array. Each module is controlled independently through Microarray Suite software using preprogrammed fluidics protocols. The scanner is a confocal laser fluorescence scanner which measures fluorescence intensity emitted by the labeled cRNA bound to the probe arrays. The computer workstation with Microarray Suite software controls the fluidics station and the scanner. Microarray Suite software can control up to eight fluidics stations using preprogrammed hybridization, wash, and stain protocols for the probe array. The software also acquires and converts hybridization intensity data into a presence/absence call for each gene using appropriate algorithms. Finally, the software detects changes in gene expression between experiments by comparison analysis and formats the output into .txt files, which can be used with other software programs for further data analysis.

The expression of a selected biomarker may also be assessed by examining gene deletion or gene amplification. Gene deletion or amplification may be measured by any one of a wide variety of protocols known in the art, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization (e.g., FISH), using an appropriately labeled probe, cytogenetic methods or comparative genomic hybridization (CGH) using an appropriately labeled probe.

Detection of Polypeptides: In another embodiment of the present invention, a polypeptide corresponding to a marker is detected. A preferred agent for detecting a polypeptide of the invention is an antibody or aptamer capable of binding to a polypeptide corresponding to a marker of the invention, preferably an antibody with a detectable label. Antibodies can be polyclonal, or more preferably, monoclonal. An intact antibody, or a fragment thereof, e.g., Fab or F(ab′)₂ can be used. The term “labeled”, with regard to the probe or antibody, is intended to encompass direct-labeling of the probe or antibody by coupling, i.e., physically linking, a detectable substance to the probe or antibody, as well as indirect-labeling of the probe or antibody by reactivity with another reagent that is directly-labeled. Examples of indirect labeling include detection of a primary antibody using a fluorescently-labeled secondary antibody and end-labeling of a DNA probe with biotin such that it can be detected with fluorescently-labeled streptavidin.

Proteins from individuals can be isolated using techniques that are well-known to those of skill in the art. The protein isolation methods employed can, e.g., be such as those described in Harlow & Lane (1988), supra. A variety of formats can be employed to determine whether a sample contains a protein that binds to a given antibody. Expression of various biomarkers in a sample can be analyzed by a number of methodologies, many of which are known in the art and understood by the skilled artisan, including but not limited to, immunohistochemical and/or Western analysis, quantitative blood based assays (as for example Serum ELISA) (to examine, for example, levels of protein expression), biochemical enzymatic activity assays, in situ hybridization, Northern analysis and/or PCR analysis of mRNAs, as well as any one of the wide variety of assays that can be performed by gene and/or tissue array analysis. Typical protocols for evaluating the status of genes and gene products are found, for example in Ausubel et al. eds., 1995, Current Protocols In Molecular Biology, Units 2 (Northern Blotting), 4 (Southern Blotting), 15 (Immunoblotting) and 18 (PCR Analysis). A skilled artisan can readily adapt known protein/antibody detection methods for use in determining whether cells express a marker of the present invention and the relative concentration of that specific polypeptide expression product in blood or other body tissues.

In such alternative methods, a sample may be contacted with an antibody specific for said biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target biomarker.

Sandwich assays are among the most useful and commonly used assays. A number of variations of the sandwich assay technique exist, and all are intended to be encompassed by the present invention. Briefly, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by the reporter molecule. By “reporter molecule”, as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, -galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

Methods of the invention further include protocols which examine the presence and/or expression of mRNAs, in a tissue or cell sample. Methods for the evaluation of mRNAs in cells are well known and include, for example, hybridization assays using complementary DNA probes (such as in situ hybridization using labeled riboprobes, Northern blot and related techniques) and various nucleic acid amplification assays (such as RT-PCR and other amplification type detection methods, such as, for example, branched DNA, SISBA, TMA and the like).

In an embodiment, the level of mRNA corresponding to the marker can be determined both by in situ and by in vitro formats in a biological sample using methods known in the art. Many expression detection methods use isolated RNA. For in vitro methods, any RNA isolation technique that does not select against the isolation of mRNA can be utilized for the purification of RNA from cells. See, e.g., Ausubel et al., Ed., Curr. Prot. Mol. Biol., John Wiley & Sons, NY (1987-1999). Additionally, large numbers of tissue samples can readily be processed using techniques well-known to those of skill in the art, such as, e.g., the single-step RNA isolation process of U.S. Pat. No. 4,843,155. The isolated mRNA can be used in hybridization or amplification assays that include, but are not limited to, Southern or Northern analyses, PCR analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, e.g., a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example, by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

Although amplification of molecules is not required in the present invention as discussed in the examples section, one of skill in the art could use amplification methods. One alternative method for determining the level of mRNA corresponding to a marker of the present invention in a sample involves the process of nucleic acid amplification, e.g., by RT-PCR (the experimental embodiment set forth in Mullis, U.S. Pat. No. 4,683,202 (1987); ligase chain reaction, self-sustained sequence replication, Guatelli et al., Proc. Natl. Acad. Sci. USA, Vol. 87, pp. 1874-1878 (1990); transcriptional amplification system, Kwoh et al., Proc. Natl. Acad. Sci. USA, Vol. 86, pp. 1173-1177 (1989); Q-Beta Replicase, Lizardi et al., Biol. Technology, Vol. 6, p. 1197 (1988); rolling circle replication, U.S. Pat. No. 5,854,033 (1988); or any other nucleic acid amplification method, followed by the detection of the amplified molecules using techniques well-known to those of skill in the art. These detection schemes are especially useful for the detection of the nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10-30 nucleotides in length and flank a region from about 50-200 nucleotides in length. Under appropriate conditions and with appropriate reagents, such primers permit the amplification of a nucleic acid molecule comprising the nucleotide sequence flanked by the primers.

For in situ methods, mRNA does not need to be isolated form the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes, such as the actin gene or epithelial cell-specific genes. This normalization allows the comparison of the expression level in one sample, e.g., a patient sample, to another sample or between samples from different sources.

Alternatively, the expression level can be provided as a relative expression level. To determine a relative expression level of a marker, the level of expression of the marker is determined for 10 or more samples of normal versus disease biological samples, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from patients who do not have the polymorphism. The choice of the cell source is dependent on the use of the relative expression level. Using expression found in normal tissues as a mean expression score aids in validating whether the marker assayed is specific (versus normal cells). In addition, as more data is accumulated, the mean expression value can be revised, providing improved relative expression values based on accumulated data.

Antibodies and Aptamers

In a preferred embodiment, the antibodies and aptamers specifically bind each component of the biomarkers described herein. The components include the nucleic acid sequences, complementary sequences, fragments, alleles, variants and gene products thereof of each component in each biomarker.

Aptamer polynucleotides are typically single-stranded standard phosphodiester DNA (ssDNA). Close DNA analogs can also be incorporated into the aptamer as described below.

A typical aptamer discovery procedure is described below:

A polynucleotide comprising a randomized sequence between “arms” having constant sequence is synthesized. The arms can include restriction sites for convenient cloning and can also function as priming sites for PCR primers. The synthesis can easily be performed on commercial instruments.

The target protein is treated with the randomized polynucleotide. The target protein can be in solution and then the complexes immobilized and separated from unbound nucleic acids by use of an antibody affinity column. Alternatively, the target protein might be immobilized before treatment with the randomized polynucleotide.

The target protein-polynucleotide complexes are separated from the uncomplexed material and then the bound polynucleotides are separated from the target protein. The bound nucleic acid can then be characterized, but is more commonly amplified, e.g. by PCR and the binding, separation and amplification steps are repeated. In many instances, use of conditions increasingly promoting separation of the nucleic acid from the target protein, e.g. higher salt concentration, in the binding buffer used in step 2) in subsequent iterations, results in identification of polynucleotides having increasingly high affinity for the target protein.

The nucleic acids showing high affinity for the target proteins are isolated and characterized. This is typically accomplished by cloning the nucleic acids using restriction sites incorporated into the arms, and then sequencing the cloned nucleic acid.

The affinity of aptamers for their target proteins is typically in the nanomolar range, but can be as low as the picomolar range. That is K_{D} is typically 1 pM to 500 nM, more typically from 1 pM to 100 nM. Aptamers having an affinity of K_{D} in the range of 1 pM to 10 nM are also useful.

Aptamer polynucleotides can be synthesized on a commercially available nucleic acid synthesizer by methods known in the art. The product can be purified by size selection or chromatographic methods.

Aptamer polynucleotides are typically from about 10 to 200 nucleotides long, more typically from about 10 to 100 nucleotides long, still more typically from about 10 to 50 nucleotides long and yet more typically from about 10 to 25 nucleotides long. A preferred range of length is from about 10 to 50 nucleotides.

The aptamer sequences can be chosen as a desired sequence, or random or partially random populations of sequences can be made and then selected for specific binding to a desired target protein by assay in vitro. Any of the typical nucleic acid-protein binding assays known in the art can be used, e.g. “Southwestern” blotting using either labeled oligonucleotide or labeled protein as the probe. See also U.S. Pat. No. 5,445,935 for a fluorescence polarization assay of protein-nucleic acid interaction.

Appropriate nucleotides for aptamer synthesis and their use, and reagents for covalent linkage of proteins to nucleic acids and their use, are considered known in the art.

A desired aptamer-protein complex, for example, aptamer-thrombin complex of the invention can be labeled and used as a diagnostic agent in vitro in much the same manner as any specific protein-binding agent, e.g. a monoclonal antibody. Thus, an aptamer-protein complex of the invention can be used to detect and quantitate the amount of its target protein in a sample, e.g. a blood sample, to provide diagnosis of a disease state correlated with the amount of the protein in the sample.

A desired aptamer-target/bait molecular complex can also be used for diagnostic imaging. In imaging uses, the complexes are labeled so that they can be detected outside the body. Typical labels are radioisotopes, usually ones with short half-lives. The usual imaging radioisotopes, such as ¹²³I, ¹²⁴I, ¹²⁵I, ¹³¹I, ^{99m}TC, ¹⁸⁶Re, ¹⁸⁸Re, ⁶⁴Cu, ⁶⁷Cu, ²¹²Bi, ²¹³Bi, ⁶⁷Ga, ⁹⁰Y, ¹¹¹In, ¹⁸F, ³H, ¹⁴C, ³⁵S or ³²P can be used. Nuclear magnetic resonance (NMR) imaging enhancers, such as gadolinium-153, can also be used to label the complex for detection by NMR. Methods and reagents for performing the labeling, either in the polynucleotide or in the protein moiety, are considered known in the art.

In a preferred embodiment, an antibody or aptamer is specific for each biomolecule of biomarker (TBB-I) comprising: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE: 5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, variants and gene products thereof.

In a preferred embodiment, an antibody or aptamer is specific for each biomolecule of biomarker (TBB-II) comprising: 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)); 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), 43934_at (G protein-coupled receptor 137), complementary sequences, fragments, alleles, variants and gene products thereof.

In a preferred embodiment, an antibody or aptamer is specific for each biomolecule of biomarker (TBB-III) comprising: 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE:5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 243766_s_at (TEA domain family member 2), and 244042_x_at complementary sequences, fragments, alleles, variants and gene products thereof.

Drug Discovery

In other preferred embodiments, the molecular signatures are useful for the identification of new drugs in the treatment of cardiovascular diseases and disorders.

Small Molecules: Small molecule test compounds or candidate therapeutic compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

Particular screening applications of this invention relate to the testing of pharmaceutical compounds in drug research. The reader is referred generally to the standard textbook “In vitro Methods in Pharmaceutical Research”, Academic Press, 1997, and U.S. Pat. No. 5,030,015). Assessment of the activity of candidate pharmaceutical compounds generally involves administering a candidate compound, determining any change in the morphology, marker phenotype and expression, or metabolic activity of the cells and function of the cells that is attributable to the compound (compared with untreated cells or cells treated with an inert compound), and then correlating the effect of the compound with the observed change.

The screening may be done, for example, either because the compound is designed to have a pharmacological effect on certain cell types, or because a compound designed to have effects elsewhere may have unintended side effects. Two or more drugs can be tested in combination (by combining with the cells either simultaneously or sequentially), to detect possible drug-drug interaction effects. In some applications, compounds are screened initially for potential toxicity (Castell et al., pp. 375-410 in “In vitro Methods in Pharmaceutical Research,” Academic Press, 1997). Cytotoxicity can be determined in the first instance by the effect on cell viability, survival, morphology, and expression or release of certain markers, receptors or enzymes. Effects of a drug on chromosomal DNA can be determined by measuring DNA synthesis or repair. [³H]thymidine or BrdU incorporation, especially at unscheduled times in the cell cycle, or above the level required for cell replication, is consistent with a drug effect. Unwanted effects can also include unusual rates of sister chromatid exchange, determined by metaphase spread. The reader is referred to A. Vickers (PP 375-410 in “In vitro Methods in Pharmaceutical Research,” Academic Press, 1997) for further elaboration.

In one embodiment of the invention, a method of identifying a candidate agent is provided said method comprising: (a) contacting a biological sample from a patient with the candidate agent and determining the level of expression of one or more biomarkers described herein; (b) determining the level of expression of a corresponding biomarker or biomarkers in an aliquot of the biological sample not contacted with the candidate agent; (c) observing the effect of the candidate agent by comparing the level of expression of the biomarker or biomarkers in the aliquot of the biological sample contacted with the candidate agent and the level of expression of the corresponding biomarker or biomarkers in the aliquot of the biological sample not contacted with the candidate agent; and (d) identifying said agent from said observed effect, wherein an at least 1%, 2%, 5%, 10% difference between the level of expression of the biomarker gene or combination of biomarker genes in the aliquot of the biological sample contacted with the candidate agent and the level of expression of the corresponding biomarker gene or combination of biomarker genes in the aliquot of the biological sample not contacted with the candidate agent is an indication of an effect of the candidate agent.

In preferred embodiments, the effects of the drug are correlated with the expression of the molecular signatures associated with a good prognosis as described in detail in the examples which follow.

In another embodiment of the invention, a candidate agent derived by the method according to the invention is provided.

In another embodiment of the invention, a pharmaceutical preparation comprising an agent according to the invention is provided.

In another preferred embodiment of the invention, a method of producing a drug comprising the steps of the method according to the invention (i) synthesizing the candidate agent identified in step (c) above or an analog or derivative thereof in an amount sufficient to provide said drug in a therapeutically effective amount to a subject; and/or (ii) combining the drug candidate the candidate agent identified in step (c) above or an analog or derivative thereof with a pharmaceutically acceptable carrier.

Vectors, Cells: In some embodiments it is desirable to express the biomolecules that comprise a biomarker, in a vector and in cells. The applications of such combinations are unlimited. The vectors and cells expressing the one or more biomolecules can be used in assays, kits, drug discovery, diagnostics, prognostics and the like. The cells can be stem cells isolated from the bone marrow as a progenitor cell, or cells obtained from any other source, such as for example, ATCC.

“Bone marrow derived progenitor cell” (BMDC) or “bone marrow derived stem cell” refers to a primitive stem cell with the machinery for self-renewal constitutively active. Included in this definition are stem cells that are totipotent, pluripotent and precursors. A “precursor cell” can be any cell in a cell differentiation pathway that is capable of differentiating into a more mature cell. As such, the term “precursor cell population” refers to a group of cells capable of developing into a more mature cell. A precursor cell population can comprise cells that are totipotent, cells that are pluripotent and cells that are stem cell lineage restricted (i.e. cells capable of developing into less than all hematopoietic lineages, or into, for example, only cells of erythroid lineage). As used herein, the term “totipotent cell” refers to a cell capable of developing into all lineages of cells. Similarly, the term “totipotent population of cells” refers to a composition of cells capable of developing into all lineages of cells. Also as used herein, the term “pluripotent cell” refers to a cell capable of developing into a variety (albeit not all) lineages and are at least able to develop into all hematopoietic lineages (e.g., lymphoid, erythroid, and thrombocytic lineages). Bone marrow derived stem cells contain two well-characterized types of stem cells. Mesenchymal stem cells (MSC) normally form chondrocytes and osteoblasts. Hematopoietic stem cells (HSC) are of mesodermal origin that normally give rise to cells of the blood and immune system (e.g., erythroid, granulocyte/macrophage, megakaryocyte and lymphoid lineages). In addition, hematopoietic stem cells also have been shown to have the potential to differentiate into the cells of the liver (including hepatocytes, bile duct cells), lung, kidney (e.g., renal tubular epithelial cells and renal parenchyma), gastrointestinal tract, skeletal muscle fibers, astrocytes of the CNS, Purkinje neurons, cardiac muscle (e.g., cardiomyocytes), endothelium and skin.

In a preferred embodiment, a method of identifying candidate therapeutic compounds comprises culturing cells expressing at least one biomolecule selected from biomarker (TBB-I) having nucleic acid sequences/biomolecules comprising: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE: 5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, variants and gene products thereof, complementary sequences, fragments, alleles, derivatives, variants, gene products or combinations thereof, with a candidate therapeutic agent; identifying candidate therapeutic agents which modulate the expression of the biomolecules and identifying a candidate therapeutic agent. Preferably, a candidate therapeutic agent comprises organic molecules, inorganic molecules, vaccines, antibodies, nucleic acid molecules, proteins, peptides and vectors expressing nucleic acid molecules.

In a preferred embodiment, a method of identifying candidate therapeutic compounds comprises culturing cells expressing at least one biomolecule selected from: biomarker (TBB-I) having nucleic acid sequences/biomolecules comprising: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2); 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE:5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, variants and gene products thereof, complementary sequences, fragments, alleles, derivatives, variants, gene products or combinations thereof.

In a preferred embodiment, a method of identifying candidate therapeutic compounds comprises culturing cells expressing at least one biomolecule selected from: biomarker (TBB-II) having nucleic acid sequences/biomolecules comprising: 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), 43934_at (G protein-coupled receptor 137), complementary sequences, fragments, alleles, derivatives, variants, gene products or combinations thereof.

In a preferred embodiment, a method of identifying candidate therapeutic compounds comprises culturing cells expressing at least one biomolecule selected from: biomarker (TBB-III) having nucleic acid sequences/biomolecules comprising: 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE: 5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 243766_s_at (TEA domain family member 2), and 244042_x_at complementary sequences, fragments, alleles, derivatives, variants, gene products or combinations thereof.

Such compounds are useful, e.g., as candidate therapeutic compounds for the treatment of heart disease, heart disorders and conditions thereof. Thus, included herein are methods for screening for candidate therapeutic compounds for the treatment of, for example, myocarditis, Coronary Heart Disease, angina, Acute Coronary Syndrome, Aortic Aneurysm and Dissection, arrhythmias, Cardiomyopathy, Congenital Heart Disease, congestive heart failure or chronic heart failure, pericarditis, and the like. The methods include administering the compound to a model of the condition, e.g., contacting a cell (in vitro) model with the compound, or administering the compound to an animal model of the condition, e.g., an animal model of a condition associated with heart disease. The model is then evaluated for an effect of the candidate compound on the clinical outcome in the model and can be considered a candidate therapeutic compound for the treatment of the condition. Such effects can include clinically relevant effects, decreased pain; increased life span; and so on. Such effects can be determined on a macroscopic or microscopic scale. Candidate therapeutic compounds identified by these methods can be further verified, e.g., by administration to human subjects in a clinical trial.

The biomolecules can be expressed from one or more vectors. A “vector” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest in gene therapy. Vectors include, for example, viral vectors (such as adenoviruses (“Ad”), adeno-associated viruses (AAV), and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

In another preferred embodiment, a vector expresses one or more biomolecules that comprise or make up TBB-I.

In another preferred embodiment, a vector expresses one or more biomolecules that comprise or make up TBB-II.

In another preferred embodiment, a vector expresses one or more biomolecules that comprise or make up TBB-III.

Kits

In another preferred embodiment, a kit is provided comprising any one or more of the biomarkers or molecular signatures comprising Tables 1, 2, and 4.

For use in the applications described or suggested above, kits or articles of manufacture are also provided by the invention. Such kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label.

The kit of the invention will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above.

The kits of the invention have a number of embodiments. A typical embodiment is a kit comprising a container, a label on said container, and a composition contained within said container; wherein the composition includes a primary antibody that binds to the biomolecules of each molecular signature and instructions for using the antibody for evaluating the presence of biomolecules in at least one type of mammalian cell. The kit can further comprise a set of instructions and materials for preparing a tissue sample and applying antibody and probe to the same section of a tissue sample. The kit may include both a primary and secondary antibody, wherein the secondary antibody is conjugated to a label, e.g., an enzymatic label.

Another embodiment is a kit comprising a container, a label on said container, and a composition contained within said container; wherein the composition includes a polynucleotide that hybridizes to a complement of the polynucleotides under stringent conditions, the label on said container indicates that the composition can be used to evaluate the presence of a molecular signature in at least one type of mammalian cell, and instructions for using the polynucleotide for evaluating the presence of biomolecule RNA or DNA in at least one type of mammalian cell.

Other optional components in the kit include, microarrays, one or more buffers (e.g., block buffer, wash buffer, substrate buffer, etc), other reagents such as substrate (e.g., chromogen) which is chemically altered by an enzymatic label, epitope retrieval solution, control samples (positive and/or negative controls), control slide(s) etc.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

Materials and Methods:

Patients: EMBs were collected from patients that were referred to Johns Hopkins Hospital between 1997 and 2006 for evaluation of cardiomyopathy (n=350). A clinical data base of patient outcome was maintained concurrently for a 10 year period beginning in 1997. All patients gave written informed consent for sample collection and medical chart abstraction. Transvenous EMBs from the right ventricular septum were obtained as previously described (Felker G M, Thompson R E, Hare J M, Hruban R H, Clemetson D E, Howard D L, Baughman K L, Kasper E K. N Engl J Med 2000 Apr. 13; 342(15):1077-84) for subsequent microarray analysis. In order to avoid possible disease specific confounding factors, only samples from patients with IDCM were selected. IDCM was a diagnosis of exclusion after extensive histological work-up without any detectable pathological signs. Within a repository of 180 IDCM samples, biopsies were selected in a case-control fashion based on the phenotypic extremes in survival of the cohort. A group with good prognosis (GP, n=25), was defined as having event free survival for at least 5 years after initial presentation with heart failure symptoms; a group with poor prognosis (PP, n=18), experienced an event within the first 2 years. Events included death (n=14), requirement for left ventricular assist device (n=2) or cardiac transplantation (n=2).

Processing of biopsies: EMBs were immediately flash frozen in liquid nitrogen for storage in a biorepository. All steps of RNA isolation and processing were performed according to MIAME guidelines (Minimum Information about a Microarray Experiment). Tissue samples (average diameter ˜2 mm) were homogenized with the MM 301 Mixer Mill (Retsch, Cat. No. 85120). Trizol reagent together with the Micro-to-Midi Total RNA Purification System (Invitrogen, Cat. No. 12183-018) was used for extraction of total RNA (success rate: 97% of samples). Concentration and integrity of total RNA was measured with the Agilent 2100 Bioanalyzer. All RNA samples exhibited intact 28S and 18S ribosomal RNA on denaturing agarose gel electrophoresis and the 260/280 nm absorbance readings fell in the acceptable range of 1.8-2.1. An average amount of 568±92 ng (SEM) total RNA was isolated and preprocessed with the Ovation Biotin RNA Amplification and Labeling System (NuGen, Cat. No. 2300-12).

Microarray hybridization: Samples were hybridized to the Human Genome U133 Plus 2.0 Array from Affymetrix without additional amplification step. The microarray experiments were judged successful when RNA isolation and microarray hybridization met all the indices of quality control as specified in the Affymetrix Guideline for Assessing Sample and Array Quality (Kittleson M M, Irizarry R A, Heidecker B, Hare J. M. Transcriptomics: Translation of Global Expression Analysis to Genomic Medicine. In: Willard H. F., Ginsburg G. S., eds. Handbook of Genomic Medicine. 1st ed. Elsevier; 2008). Average background and noise of all chips registered within acceptable ranges and hybridization efficiencies were similar for all samples.

Statistical Analysis: Raw intensity values from microarray hybridization were normalized with Robust Multiarray Average (RMA) implemented in the R package for statistical computing (available at www.R-project.org). In the next step, Significance Analysis of Microarrays (SAM) (Tusher V G, et al. Proc Natl Acad Sci USA 2001 Apr. 24; 98(9):5116-21) was used to identify phenotype specific differences in gene expression. SAM defines significance with the q-value, an adjusted p-value for multiple comparisons. For the development of a TBB, Prediction Analysis of Microarrays (PAM) (Tibshirani R, et al. Proc Natl Acad Sci USA 2002 May 14; 99(10):6567-72) was used to create a classifier in a training set (containing ⅔ of the data, n=29), with subsequent validation in a test set (containing ⅓ of the data, n=14)(Kittleson M M, et al. Circulation 2004 Nov. 30; 110(22):3444-51). PAM is a software that allows to find the minimum number of genes necessary to create a phenotype specific “nearest shrunken centroid” for classification (Tibshirani R, et al. 2002, supra). This was done by a balanced 10-fold cross validation in a training set, which enables one to choose a threshold that minimizes classification errors. Overall accuracy of the discovered transcriptomic biomarker was assessed after 50 random partitions. To test for balanced baseline conditions of our cohort with good vs. poor prognosis, a student t-test for numerical and Fisher's exact test for categorical variables was used.

### Example 1

### Transcriptomic Biomarker

To identify this biomarker (gene signature) heart samples were collected from patients undergoing heart biopsy at an early stage of heart failure, originating from either myocarditis or idiopathic cardiomyopathy, and stored them in a biorepository over 10 years.

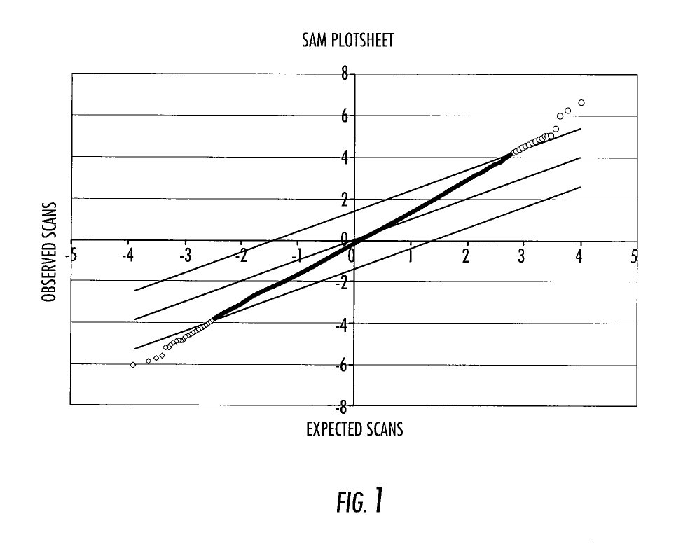

Endomyocardial biopsy samples from patients with myocarditis or idiopathic cardiomyopathy have been collected at the Johns Hopkins Hospital between 1997-2004 and stored in liquid nitrogen. The biorepository was reviewed and biopsy samples from 19 patients with myocarditis and 42 patients with idiopathic cardiomyopathy were chosen. Myocarditis was diagnosed from endomyocardial biopsy samples by immunohistochemistry. The MIAME (Minimum information about a microarray experiment) guidelines were followed for all experiments. The tissue was homogenized with the MM301 instrument from Retsch and total RNA was isolated with the Micro-to-Midi Total RNA purification system from Invitrogen. Microarray analysis of total RNA was performed with the Human Genome U133 Plus 2.0 Array from Affymetrix. In all samples, both RNA isolation and microarray hybridization met all indices of quality control as specified in the Affymetrix Guideline for Assessing Sample and Array Quality. Raw expression values of all microarray chips were preprocessed with Robust Multiarray Average (RMA) in R. We performed Significance Analysis of Microarrays (SAM) on a training set of 28 samples (11 samples from myocarditis, 17 samples from idiopathic cardiomyopathy) to select a subset of 122 differentially expressed genes (FC >1.2, q=0%). These 122 contained 38 genes that were overexpressed and 84 genes were downregulated in patients with myocarditis (FIG. 1).

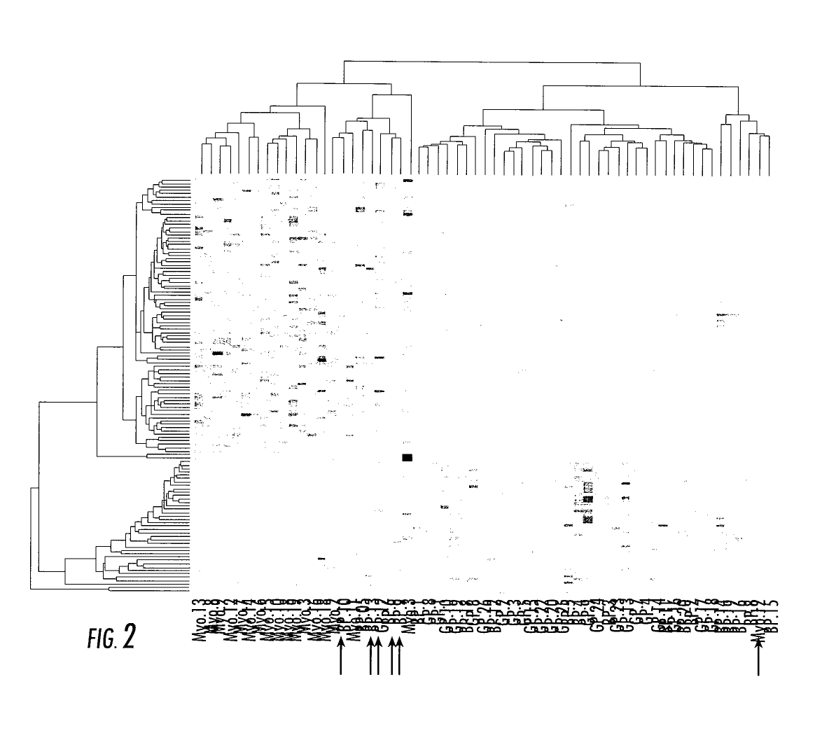

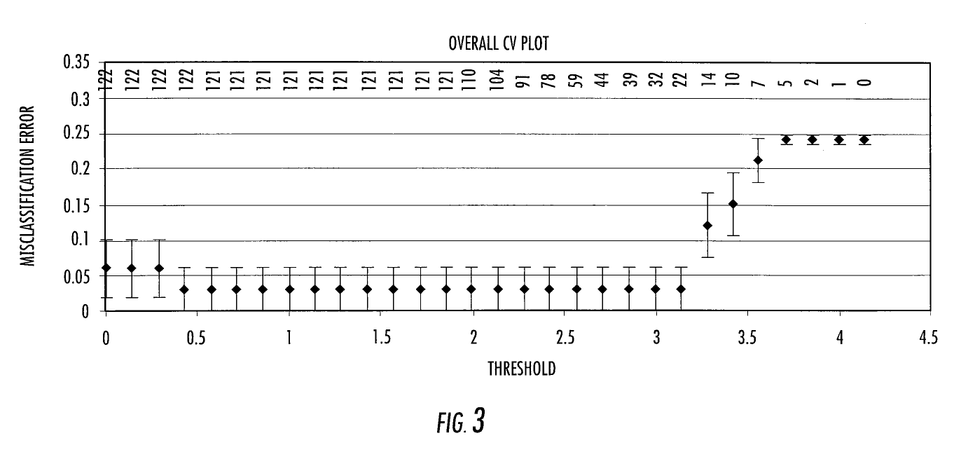

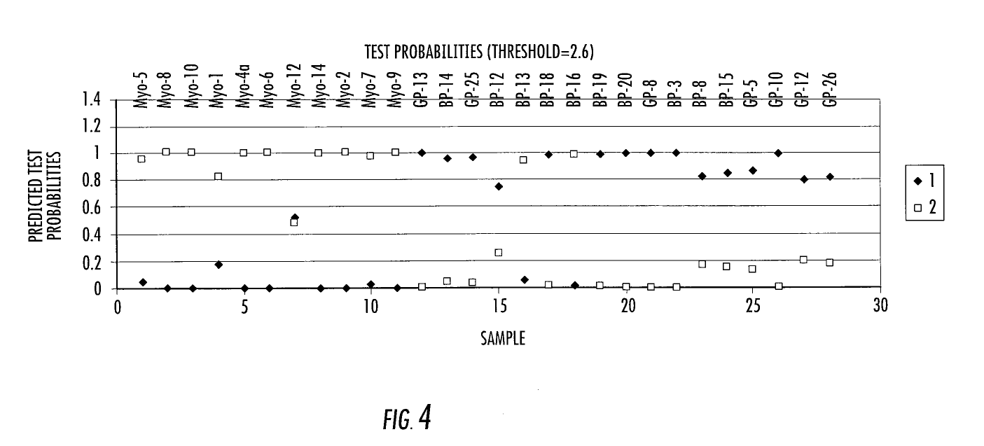

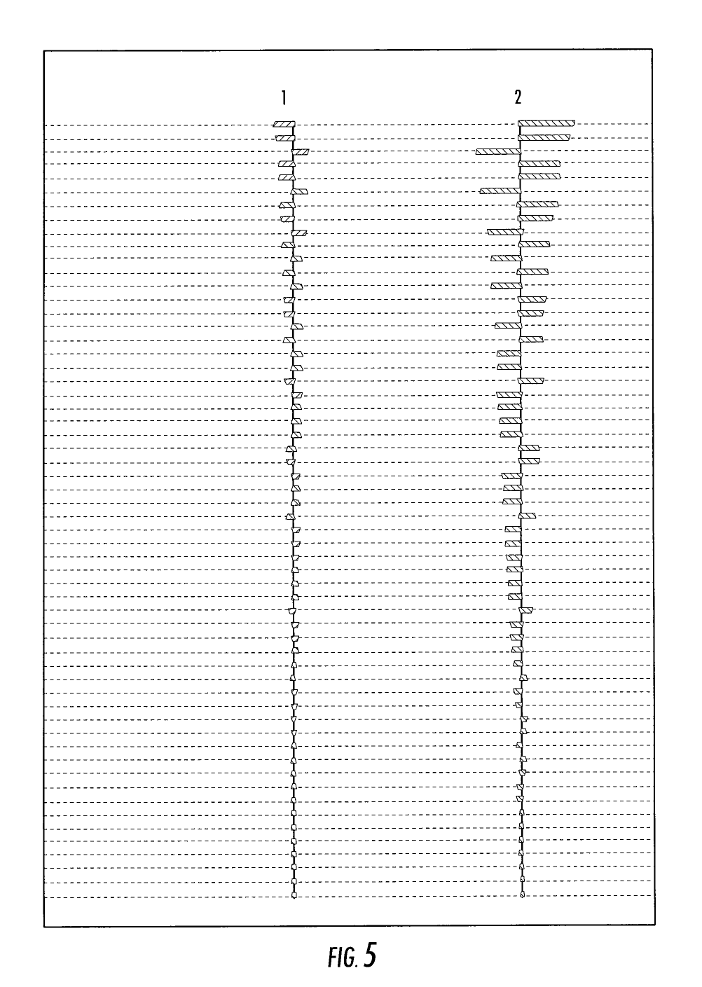

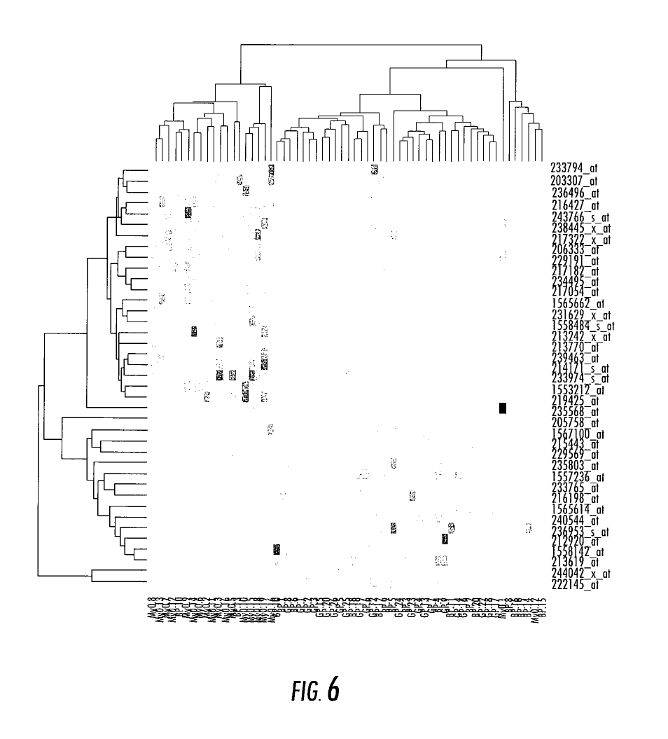

With this predefined subset of 122 genes, a heatmap was created including all samples of the study (n=61) standardized by mean levels of expression (FIG. 2, Table 1, Table 2). A heatmap is a classification method of unsupervised machine learning. Each row represents one of the 122 genes, and each column is a patient sample. A red cell depicts an underexpressed gene in a given patient relative to the average gene expression in all patients, while a blue cell denotes an overexpressed gene. The dendrogram at the top is an unsupervised hierarchical clustering algorithm that divides samples into groups based on the similarity of the gene expression profiles. This algorithm performed with 77% sensitivity and 97% specificity. To increase predictive power, the same subset of 122 genes for a “nearest shrunken centroid” classification approach with Prediction Analysis of Microarrays (PAM) implemented in R was used. After training of the classifier (supervised clustering) in a subset of 11 samples of patients with myocarditis and 17 samples of patients with idiopathic cardiomyopathy, a cluster of 39 genes that identified myocarditis with 90% sensitivity and 88% specificity was found (FIG. 3, FIG. 4, FIG. 5, Table 3, Table 4). To test if the predictive power of the discovered transcriptomic biomarker could be increased, heatmap with the new subset of 39 candidate genes was created (FIG. 6). While the first heatmap of 122 genes produced 6 misclassified samples, only 4 samples were classified incorrectly by this algorithm (sensitivity: 89%, specificity: 95%). With this extremely powerful diagnostic method of gene expression profiling, even patients that do not present with characteristic clinical signs of myocarditis, will be detected at a very early stage of the disease and treated appropriately. The application of this highly sensitive biomarker will significantly reduce the number of cases that undergo a fatal course of myocarditis in the future. In addition this biomarker will allow appropriate use of therapies specifically for myocarditis.

Table 1 shows the 38 genes that were significantly upregulated in patients with myocarditis versus idiopathic cardiomyopathy. First column contains the ID from Affymetrix for each transcript.

TABLE 1
38 significantly upregulated genes in patients with myocarditis (q = 0.1%; FC > 1.2)

| Probe Set ID | Gene Title | GO Biological Process Description |
|---|---|---|
| 1553145_at | hypothetical protein FLJ39653 | — |
| 1553575_at | — | — |
| 1557236_at | apolipoprotein L, 6 | regulation of transcription, DNA-dependent /// lipid transport |
| 1558142_at | trinucleotide repeat containing | — |
| | 6B | |
| 1560752_at | F-box and WD-40 domain | ubiquitin cycle /// protein modification /// proteolysis |
| | protein 2 | |
| 1565614_at | Zinc finger protein 337 | transcription /// regulation of transcription, DNA-dependent |
| 1567100_at | Dachshund homolog 1 | transcription /// regulation of transcription, DNA-dependent |
| | (Drosophila) | |
| 200068_s_at | calnexin /// calnexin | angiogenesis /// protein folding /// protein secretion |
| 201031_s_at | heterogeneous nuclear | mRNA processing /// RNA splicing /// RNA processing |
| | ribonucleoprotein H1 (H) | |
| 202646_s_at | cold shock domain containing | regulation of transcription, DNA-dependent /// male gonad development |
| | E1, RNA-binding | |
| 205758_at | CD8a molecule /// CD8a | immune response/// antigen processing and presentation /// T cell activation /// |
| | molecule | cytotoxic T cell differentiation /// immune response |
| 206188_at | zinc finger protein 623 | transcription /// regulation of transcription, DNA-dependent |
| 212637_s_at | WW domain containing E3 | signal transduction/// T cell differentiation ///entry of virus into host cell |
| | ubiquitin protein ligase 1 | |
| 212920_at | — | — |
| 213317_at | chloride intracellular channel 5 | ion transport /// chloride transport /// chloride transport /// pregnancy /// transport /// |
| | | transport |
| 213619_at | Heterogeneous nuclear | RNA splicing |
| | ribonucleoprotein H1 (H) | |
| 215443_at | thyroid stimulating hormone | signal transduction /// G-protein signaling |
| | receptor | |
| 216198_at | activating transcription factor 7 | DNA methylation |
| | interacting protein | |
| 217870_s_at | cytidylate kinase | pyrimidine ribonucleotide biosynthesis |
| 218087_s_at | sorbin and SH3 domain | transport /// insulin receptor signaling pathway |
| | containing 1 | |
| 222145_at | CDNA: FLJ23572 fis, clone | — |
| | LNG12403 | |
| 223577_x_at | PRO1073 protein | — |
| 224321_at | transmembrane protein with | — |
| | EGF-like and two follistatin-like | |
| | domains 2 | |
| 224373_s_at | IQ motif and WD repeats 1 | apoptosis /// signal transduction /// development /// ATP synthesis coupled electron |
| | | transport |
| 224644_at | CDNA clone IMAGE: 5278517 | — |
| 226173_at | ornithine aminotransferase-like 1 | — |
| 226773_at | CDNA FLJ35131 fis, clone | — |
| | PLACE6008824 | |
| 226880_at | nuclear casein kinase and cyclin- | — |
| | dependent kinase substrate 1 | |
| 228980_at | ring finger and FYVE-like | ubiquitin cycle /// apoptosis |
| | domain containing 1 | |
| 229569_at | CDNA clone IMAGE: 5263455 | — |
| 231735_s_at | PRO1073 protein | — |
| 233765_at | Hypothetical LOC197135 | — |
| 235803_at | Cytokine receptor-like factor 3 | — |
| 236131_at | CDNA clone IMAGE: 6622963 | — |
| 236953_s_at | similar to RIKEN cDNA | — |
| | 8030451K01 | |
| 240544_at | Zinc finger, AN1-type domain 3 | — |
| 240971_x_at | Cullin 4A | G1/S transition of mitotic cell cycle /// DNA repair /// ubiquitin cycle /// cell cycle /// |
| | | cell cycle arrest |
| 244042_x_at | Similar to retinoic acid receptor | — |
| | responder (tazarotene induced) 2 | |

Table 2 shows 84 genes that were significantly downregulated in patients with myocarditis versus patients with idiopathic cardiomyopathy (FC >1.2, q=0%).

TABLE 2
84 significantly downregulated genes in myocarditis (FC > 1.2, q = 0.1%))

| Probe Set ID | Gene Title | GO Biological Process Description |
|---|---|---|
| 1552419_s_at | tubulin tyrosine ligase-like | protein modification |
| | family, member 10 | |
| 1553212_at | keratin 78 | — |
| 1555124_at | hypothetical protein | — |
| | MGC40574 | |
| 1556192_x_at | Metastasis suppressor 1 | cell motility |
| 1556320_at | Stomatin (EPB72)-like 1 | biological_process |
| 1556510_at | CDNA clone IMAGE: 4796864 | — |
| 1558484_s_at | leucine rich repeat containing | — |
| | 27 | |
| 1565662_at | Mucin 6, oligomeric | maintenance of gastrointestinal epithelium |
| | mucus/gel-forming | |
| 1567410_at | zinc finger protein 135 | regulation of transcription, DNA-dependent |
| 1568513_x_at | Protease, serine, 1 (trypsin 1) | collagen catabolism, proteolysis, collagen catabolism, immune response |
| 1570408_at | Serine/threonine kinase 24 | protein amino acid phosphorylation, signal transduction |
| | (STE20 homolog, yeast) | |
| 203307_at | guanine nucleotide binding | signal transduction |
| | protein-like 1 | |
| 204581_at | CD22 molecule, myelin | immune response, cell adhesion, antimicrobial humoral response (sensu Vertebrata) |
| | associated glycoprotein | |
| 205586_x_at | VGF nerve growth factor | biological_process |
| | inducible | |
| 206333_at | musashi homolog 1 | nervous system development |
| | (Drosophila) | |
| 207004_at | B-cell CLL/lymphoma 2 | regulation of progression through cell cycle, humoral immune response |
| 210059_s_at | mitogen-activated protein | regulation of translation, protein amino acid phosphorylation |
| | kinase 13 | |
| 210228_at | colony stimulating factor 2 | cellular defense response |
| | (granulocyte-macrophage) | |
| 210384_at | protein arginine | protein amino acid methylation |
| | methyltransferase 2 | |
| 210923_at | solute carrier family 1 | dicarboxylic acid transport |
| | (glutamate transporter), | |
| | member 7 | |
| 211024_s_at | thyroid transcription factor 1 | neuron migration |
| | /// thyroid transcription factor 1 | |
| 211062_s_at | carboxypeptidase Z /// | proteolysis, Wnt receptor signaling pathway |
| | carboxypeptidase Z | |
| 211096_at | pre-B-cell leukemia | regulation of transcription, DNA-dependent |
| | transcription factor 2 | |
| 211181_x_at | runt-related transcription factor | skeletal development, hemopoiesis |
| | 1 (acute myeloid leukemia 1; | |
| | aml1 oncogene) | |
| 211710_x_at | ribosomal protein L4 /// | protein biosynthesis, metabolism |
| | ribosomal protein L4 | |
| 213096_at | transmembrane and coiled-coil | — |
| | domain family 2 | |
| 213121_at | small nuclear | mRNA processing |
| | ribonucleoprotein 70 kDa | |
| | polypeptide (RNP antigen) | |
| 213242_x_at | KIAA0284 | — |
| 213568_at | odd-skipped related 2 | — |
| | (Drosophila) | |
| 213770_at | kinase suppressor of ras 1 | protein amino acid phosphorylation |
| 214171_s_at | U2 small nuclear RNA | mRNA processing |
| | auxiliary factor 2 | |
| 216116_at | NCK interacting protein with | NLS-bearing substrate import into nucleus |
| | SH3 domain | |
| 216427_at | CDNA: FLJ22786 fis, clone | — |
| | KAIA2150 | |
| 216820_at | — | — |
| 217054_at | CDNA FLJ39484 fis, clone | — |
| | PROST2014925 | |
| 217180_at | Hypothetical protein similar to | immune response |
| | KIAA0187 gene product | |
| 217182_at | mucin 5AC, oligomeric | cell adhesion |
| | mucus/gel-forming | |
| 217322_x_at | — | — |
| 217430_x_at | collagen, type I, alpha 1 | skeletal development |
| 219070_s_at | motile sperm domain | heart development |
| | containing 3 | |
| 219425_at | sulfotransferase family 4A, | lipid metabolism |
| | member 1 | |
| 221663_x_at | histamine receptor H3 | G-protein coupled receptor protein signaling pathway |
| 221684_s_at | Nyctalopin | response to stimulus |
| 223974_at | hypothetical protein | — |
| | MGC11082 | |
| 226640_at | diacylglycerol lipase beta | lipid catabolism, lipid metabolism |
| 228074_at | hypothetical protein | — |
| | LOC162073 | |
| 229191_at | tubulin folding cofactor D | beta-tubulin folding |
| 229257_at | KIAA1856 protein | — |
| 229335_at | immunoglobulin superfamily, | — |
| | member 4C | |
| 229358_at | Indian hedgehog homolog | skeletal development, patterning of blood vessels |
| | (Drosophila) | |
| 230341_x_at | ADAM metallopeptidase with | proteolysis, integrin-mediated signaling pathway |
| | thrombospondin type 1 motif, | |
| | 10 | |
| 230693_at | ATPase, Ca++ transporting, | calcium ion transport, regulation of striated muscle contraction |
| | cardiac muscle, fast twitch 1 | |
| 230768_at | FERM, RhoGEF and | neuron remodeling |
| | pleckstrin domain protein 2 | |
| 231510_at | GLI-Kruppel family member | transcription from RNA polymerase II promoter |
| | GLI2 | |
| 231629_x_at | Kallikrein-related peptidase 3 | proteolysis, negative regulation of angiogenesis |
| 231998_at | — | — |
| 233794_at | Single stranded DNA binding | regulation of transcription, DNA-dependent |
| | protein 3 | |
| 233974_s_at | family with sequence | — |
| | similarity 129, member B | |
| 234495_at | kallikrein-related peptidase 15 | proteolysis |
| 234637_at | keratin associated protein 4-5 | — |
| 234881_at | — | — |
| 235568_at | chromosome 19 open reading | — |
| | frame 59 | |
| 235600_at | Transcribed locus | — |
| 236496_at | degenerative spermatocyte | lipid metabolism |
| | homolog 2, lipid desaturase | |
| | (Drosophila) | |
| 237087_at | Chromosome 14 open reading | — |
| | frame 105 | |
| 237144_at | Latent transforming growth | skeletal development, transforming growth factor beta receptor signaling pathway |
| | factor beta binding protein 3 | |
| 237398_at | Transcribed locus | — |
| 237547_at | Hypothetical protein | — |
| | LOC728730 | |
| 237679_at | tripartite motif-containing 66 | negative regulation of transcription, DNA-dependent |
| 238267_s_at | — | — |
| 238445_x_at | mannosyl (alpha-1,6-)- | — |
| | glycoprotein beta-1,6-N- | |
| | acetyl- | |
| | glucosaminyltransferase, | |
| | isozyme B | |
| 239026_x_at | centaurin, gamma 3 | regulation of transcription, DNA-dependent |
| 239463_at | Transcribed locus | — |
| 239756_at | MAD1 mitotic arrest deficient- | mitosis checkpoint |
| | like 1 (yeast) | |
| 240039_at | Transcribed locus /// | — |
| | Transcribed locus | |
| 240147_at | hypothetical protein | — |
| | MGC11257 | |
| 240517_at | Cystathionine-beta-synthase | cysteine biosynthesis from serine |
| 241270_at | Rhomboid 5 homolog 2 | — |
| | (Drosophila) | |
| 241431_at | — | — |
| 242365_at | Coiled-coil domain containing | — |
| | 32 | |
| 243297_at | Vacuolar protein sorting 13 | protein localization, cell cycle, mitosis |
| | homolog D (S. cerevisiae) | |
| 243497_at | Transcribed locus | — |
| 243766_s_at | TEA domain family member 2 | transcription, regulation of transcription, DNA-dependent |
| 43934_at | G protein-coupled receptor 137 | biological_process |

Table 3 shows the prediction results of the test set (n=28): 39 genes signature was used. The sample labels are listed above the row highlighted in red, followed by the predicted classes. Myocarditis (Myo) samples were assigned to class 2—idiopathic cardiomyopathy samples (GP, BP) were assigned to class 1. 25 samples were correctly classified (probabilities between 75% and 99%). Only three samples were misclassified (with probabilities between 48% and 99%). Predicted probabilities are listed for each class in the last two lines.

TABLE 3
Prediction for Threshold = 2.6
Settings Name: Settings4

| | | | |
|---|---|---|---|
| Offset Quantile | 50 | Offset Value | 0.257958 |
| | both | RNG Seed | 420473 |

Prior Distribution (Sample Prior)

| | | |
|---|---|---|
| Class | 1 | 2 |
| Prob. | 0.757576 | 0.242424242 |

Actual, Predicted Classes and Predicted Posterior Probabilities

| | | | | | | | | | | | | |
|---|---|---|---|---|---|---|---|---|---|---|---|---|
| Sample | Myo-5 | Myo-8 | Myo-10 | Myo-1 | Myo-4a | Myo-6 | Myo-12 | Myo-14 | Myo-2 | Myo-7 | Myo-9 | GP-13 |
| Labels | | | | | | | | | | | | |
| Class | [symbol] | [symbol] | [symbol] | [symbol] | [symbol] | [symbol] | [symbol] | [symbol] | [symbol] | [symbol] | [symbol] | [symbol] |
| Labels | | | | | | | | | | | | |
| Pre- | 2 | 2 | 2 | 2 | 2 | 2 | 1 | 2 | 2 | 2 | 2 | 1 |
| dicted | | | | | | | | | | | | |
| Class | | | | | | | | | | | | |
| Labels | | | | | | | | | | | | |
| 1 | 0.044735755 | 0.00317 | 0.000372279 | 0.173628 | 0.00064 | 0.000536 | 0.51331 | 0.000157 | 0.00408 | 0.021898 | 0.002194 | 0.999853 |
| 2 | 0.955264245 | 0.99683 | 0.999627721 | 0.826372 | 0.99936 | 0.999464 | 0.48669 | 0.999843 | 0.99592 | 0.978102 | 0.997806 | 0.000147 |

| | | | | | | | | | | | |
|---|---|---|---|---|---|---|---|---|---|---|---|
| BP-14 | GP-25 | BP-12 | BP-13 | BP-18 | BP-16 | BP-19 | BP-20 | GP-8 | BP-3 | BP-8 | BP-15 |
| [symbol] | [symbol] | [symbol] | [symbol] | [symbol] | [symbol] | [symbol] | [symbol] | [symbol] | [symbol] | [symbol] | [symbol] |
| 1 | 1 | 1 | 2 | 1 | 2 | 1 | 1 | 1 | 1 | 1 | 1 |
| 0.961375 | 0.96379 | 0.753742 | 0.06179 | 0.980899 | 0.017053 | 0.990345 | 0.998768 | 0.996078 | 0.999488 | 0.821228 | 0.850691 |
| 0.038625 | 0.03621 | 0.246258 | 0.93821 | 0.019101 | 0.982947 | 0.009655 | 0.001232 | 0.003922 | 0.000512 | 0.178772 | 0.149309 |

| | | | |
|---|---|---|---|
| GP-5 | GP-10 | GP-12 | GP-26 |
| [symbol] | [symbol] | [symbol] | [symbol] |
| 1 | 1 | 1 | 1 |
| 0.867616 | 0.998565 | 0.797331 | 0.814156 |
| 0.132384 | 0.001435 | 0.202669 | 0.185844 |

[symbol]

indicates data missing or illegible when filed

Table 4: 39 genes transcriptomic biomarker for detection of patients with myocarditis: First column contains the IDs from Affymetrix for every gene. Annotations for biological function were derived from the gene ontology database.

TABLE 4
39 genes biomarker to detect myocarditis

| Probe Set ID | Gene Title | GO Biological Process Description |
|---|---|---|
| 1553212_at | keratin 78 | — |
| 1557236_at | apolipoprotein L, 6 | regulation of transcription, DNA-dependent /// lipid |
| | | transport /// lipoprotein metabolism |
| 1558142_at | trinucleotide repeat containing 6B | — |
| 1558484_s_at | leucine rich repeat containing 27 | — |
| 1565614_at | Zinc finger protein 337 | regulation of transcription, DNA-dependent |
| 1565662_at | Mucin 6, oligomeric mucus/gel-forming | maintenance of gastrointestinal epithelium |
| 1567100_at | Dachshund homolog 1 (Drosophila) | regulation of transcription, DNA-dependent |
| 203307_at | guanine nucleotide binding protein-like 1 | signal transduction |
| 205758_at | CD8a molecule /// CD8a molecule | antigen processing and presentation, cytotoxic T cell |
| | | differentiation immune response |
| 206333_at | musashi homolog 1 (Drosophila) | nervous system development |
| 212920_at | — | — |
| 213242_x_at | KIAA0284 | — |
| 213619_at | Heterogeneous nuclear ribonucleoprotein | mRNA processing |
| | H1 (H) | |
| 213770_at | kinase suppressor of ras 1 | protein amino acid phosphorylation |
| 214171_s_at | U2 small nuclear RNA auxiliary factor 2 | nuclear mRNA splicing, via spliceosome |
| 215443_at | thyroid stimulating hormone receptor | signal transduction, G-protein signaling, coupled to |
| | | cyclic nucleotide second messenger |
| 216198_at | activating transcription factor 7 | DNA methylation |
| | interacting protein | |
| 216427_at | CDNA: FLJ22786 fis, clone KAIA2150 | — |
| 217054_at | CDNA FLJ39484 fis, clone | — |
| | PROST2014925 | |
| 217182_at | mucin 5AC, oligomeric mucus/gel- | cell adhesion |
| | forming | |
| 217322_x_at | — | — |
| 219425_at | sulfotransferase family 4A, member 1 | lipid metabolism |
| 222145_at | CDNA: FLJ23572 fis, clone LNG12403 | — |
| 229191_at | tubulin folding cofactor D | protein folding |
| 229569_at | CDNA clone IMAGE: 5263455 | — |
| 231629_x_at | Kallikrein-related peptidase 3 | proteolysis, negative regulation of angiogenesis |
| 233765_at | Hypothetical LOC197135 | — |
| 233794_at | Single stranded DNA binding protein 3 | regulation of transcription, DNA-dependent |
| 233974_s_at | family with sequence similarity 129, | — |
| | member B | |
| 234495_at | kallikrein-related peptidase 15 | proteolysis, proteolysis |
| 235568_at | chromosome 19 open reading frame 59 | — |
| 235803_at | Cytokine receptor-like factor 3 | |
| 236496_at | degenerative spermatocyte homolog 2, | lipid metabolism |
| | lipid desaturase (Drosophila) | |
| 236953_s_at | similar to RIKEN cDNA 8030451K01 | — |
| 238445_x_at | mannosyl (alpha-1,6-)-glycoprotein beta- | — |
| | 1,6-N-acetyl-glucosaminyltransferase, | |
| | isozyme B | |
| 239463_at | Transcribed locus | — |
| 240544_at | Zinc finger, AN1-type domain 3 | — |
| 243766_s_at | TEA domain family member 2 | regulation of transcription, DNA-dependent |
| 244042_x_at | Similar to retinoic acid receptor responder | — |
| | (tazarotene induced) 2 | |

Summary: The risk score from a prognostic molecular signature predicts clinical outcome of patients with IP with high accuracy.

The value of this prognostic test was dramatically improved for patients with a good prognosis (GP) or poor prognosis (PP). Good prognosis (GP): Event free survival for at least 5 years after presentation of heart failure (HF), n=25. Poor prognosis (PP): Event within first 2 years after presentation of HF (LVAD, tx, death), n=18.

Transcriptomic biomarker developed in IDCM is generalizable to secondary noninflammatory cardiomyopathies.

### Other Embodiments

It is to be understood that while the invention has been described in conjunction with the detailed description thereof, the foregoing description is intended to illustrate and not limit the scope of the invention. Other aspects, advantages, and modifications are within the scope of the following claims.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the disclosure will allow the reader to quickly ascertain the nature of the technical disclosure. It is submitted with the understanding that it will not be used to interpret or limit the scope or meaning of the following claims.

What is claimed is:

1. A biomarker for the diagnosis of myocarditis comprising markers: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE:5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, derivatives variants and/or gene products thereof.

2. The biomarker of claim 1, wherein the biomarker comprises markers: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE:5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), and 244042_x_at (Similar to retinoic acid receptor responder (tazarotene induced) 2).

3. A method of diagnosing myocarditis in a patient comprising:
determining a marker profile from a sample taken from a patient;
correlating the marker profile obtained from the patient's sample to a biomarker diagnostic of myocarditis; and,
diagnosing myocarditis in a patient.

4. The method of claim 3, wherein the biomarker diagnostic of myocarditis comprises markers:
1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE:5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, derivatives, variants and/or gene products thereof.

5. The method of claim 3, wherein the biomarker diagnostic of myocarditis comprises an expression profile comprising markers:
1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE:5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), and 244042_x_at (Similar to retinoic acid receptor responder (tazarotene induced) 2).

6. The method of claim 3, wherein the markers are detected by microarray assays, immunoassays, PCR or mass spectrometry.

7. The method of claim 3, wherein the marker expression profile is detected by microarray assays, immunoassays, PCR or mass spectrometry.

8. The method of claim 5, wherein phenotypic specific differences in marker expression profiles are identified by significance analysis of microarrays, wherein significance is defined with a q-value and multiple comparisons comprise an adjusted p-value.

9. The method of claim 5, wherein a predictive value of the marker expression profile is determined by a prediction analysis of microarrays comprising a nearest shrunken centroid analysis.

10. The method of claim 5, wherein a predictive value of the polynucleotide or polypeptide expression profile is determined by a heatmap analysis.

11. A method of diagnosing and differentiating between myocarditis and idiopathic cardiomyopathy comprising:
determining a marker profile from a sample taken from a patient;
comparing the marker profile obtained from the patient's sample to a biomarker diagnostic of myocarditis; and,
diagnosing and differentiating between myocarditis and idiopathic cardiomyopathy in a patient.

12. The method of claim 11, wherein the biomarker comprises markers:
1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE:5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE: 6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, derivatives, variants and/or gene products thereof.

13. The method of claim 11, wherein the biomarker comprises markers: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE:5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), and 244042_x_at (Similar to retinoic acid receptor responder (tazarotene induced) 2).

14. The method of claim 11, wherein detection in a sample of at least one marker is diagnostic of myocarditis versus idiopathic cardiomyopathy.

15. The method of claim 11, wherein detection in a sample of a plurality of markers is diagnostic of myocarditis versus idiopathic cardiomyopathy.

16. The method of claim 11, wherein the markers are detected by microarray assays, immunoassays, PCR or mass spectrometry.

17. The method of claim 11, wherein phenotypic specific differences in marker expression are identified by significance analysis of microarrays, wherein significance is defined with a q-value and multiple comparisons comprise an adjusted p-value.

18. The method of claim 11, wherein a predictive value of marker expression is determined by a prediction analysis of microarrays comprising a nearest shrunken centroid analysis.

19. The method of claim 11, wherein a predictive value of marker expression is determined by a heatmap analysis.

20. A biomarker for the diagnosis of myocarditis comprising 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase; isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), 43934_at (G protein-coupled receptor 137), complementary sequences, fragments, alleles, derivatives, variants and/or gene products thereof.

21. The biomarker of claim 20, wherein the biomarker comprises markers: 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), and 43934_at (G protein-coupled receptor 137).

22. A method of detecting and diagnosing myocarditis comprising:
obtaining a marker expression profile from a sample taken from a patient;
comparing the marker expression profile obtained from the patient's sample to a biomarker; and,
diagnosing myocarditis in a patient.

23. The method of claim 22, wherein the biomarker comprises markers:
1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), 43934_at (G protein-coupled receptor 137), complementary sequences, fragments, alleles, derivatives, variants and/or gene products thereof.

24. The method of claim 22, wherein the biomarker comprises markers: 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), and 43934_at (G protein-coupled receptor 137).

25. The method of claim 22, wherein a decreased expression or down-regulation of at least one marker as compared to controls is diagnostic of myocarditis.

26. The method of claim 22, wherein a decreased expression or down-regulation of a plurality of markers as compared to controls is diagnostic of myocarditis.

27. The method of claim 22, wherein the marker expression profile is detected by microarray assays, immunoassays, PCR or mass spectrometry.

28. The method of claims 22, wherein the markers are detected by microarray assays, immunoassays, PCR or mass spectrometry.

29. The method of claim 22, wherein phenotypic specific differences in marker expression are identified by significance analysis of microarrays, wherein significance is defined with a q-value and multiple comparisons comprise an adjusted p-value.

30. The method of claim 22, wherein a predictive value of marker expression is determined by a prediction analysis of microarrays comprising a nearest shrunken centroid analysis.

31. The method of claim 22, wherein a predictive value of marker expression is determined by a heatmap analysis.

32. A biomarker for the detection and diagnosis of myocarditis comprising markers: 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE:5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 243766_s_at (TEA domain family member 2), 244042_x_at, complementary sequences, fragments, alleles, derivatives, variants and/or gene products thereof.

33. The biomarker of claim 32, wherein the biomarker comprises markers: 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE:5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 243766_s_at (TEA domain family member 2), and 244042_x_at.

34. A method of detecting and diagnosing myocarditis comprising:
obtaining a marker expression profile from a sample taken from a patient;
comparing the marker expression profile obtained from the patient's sample to a biomarker; and,
diagnosing myocarditis in a patient.

35. The method of claim 34, wherein the biomarker comprises markers: 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE:5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 243766_s_at (TEA domain family member 2), 244042_x_at, complementary sequences, fragments, alleles, derivatives, variants and/or gene products thereof.

36. The method of claim 34, wherein the biomarker comprises markers: 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE:5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 243766_s_at (TEA domain family member 2), and 244042_x_at.

37. The method of claim 34, wherein the marker expression profile is detected by microarray assays, immunoassays, PCR or mass spectrometry.

38. The method of claim 37, wherein phenotypic specific differences in marker expression are identified by significance analysis of microarrays, wherein significance is defined with a q-value and multiple comparisons comprise an adjusted p-value.

39. The method of claim 34, wherein a predictive value of marker expression is determined by a prediction analysis of microarrays comprising a nearest shrunken centroid analysis.

40. The method of claim 34, wherein a predictive value of marker expression is determined by a heatmap analysis.

41. An antibody or aptamer specific for each gene sequence comprising nucleic acid sequences/biomolecules comprising: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE:5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, derivatives, variants and/or gene products thereof.

42. A biochip comprising nucleic acid sequences: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE: 5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), 244042_x_at (Similar to retinoic acid receptor responder (tazarotene induced) 2), complementary sequences, fragments, alleles, variants and gene products thereof, complementary sequences, fragments, alleles, variants and/or gene products thereof.

43. The biochip of claim 42, wherein the biochip comprises at least ten nucleic sequences, complementary sequences, fragments; alleles, variants and gene products thereof.

44. A biochip comprising nucleic acid sequences: 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), 43934_at (G protein-coupled receptor 137), complementary sequences, fragments, alleles, derivatives, variants and/or gene products thereof.

45. An antibody or aptamer specific for each gene sequence comprising nucleic acid sequences/biomolecules comprising: 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at (kinase suppressor of ras 1), 214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), 43934_at (G protein-coupled receptor 137), complementary sequences, fragments, alleles, derivatives, variants and/or gene products thereof.

46. A method of diagnosing myocarditis, comprising:
quantifying in a biological sample from a patient a biomarker selected from the group consisting of transcriptomic based biomarker-I (TBB-I), transcriptomic based biomarker-II (TBB-II) and transcriptomic based biomarker-III (TBB-III) or combinations thereof; and,
diagnosing myocarditis.

47. The method of claim 46, wherein the biomarker is identified from a patient by isolating nucleic acids obtained from a biological sample.

48. The method of claim 46, wherein the biomarkers are detected by microarray assays, immunoassays, PCR or mass spectrometry.

49. The method of claim 46, wherein the nucleic acids are hybridized to the biochip and raw intensity values from microarray hybridization are normalized and phenotype specific differences in gene expression are identified.

50. The method of claim 46, wherein a phenotype specificity is identified by creating a classifier in a training set comprising about 66% of data obtained, with subsequent validation in a test set comprising about 33% of data obtained and defining a phenotype specific nearest shrunken centroid for classification.

51. The method of claim 46, wherein the phenotype specific nearest shrunken centroid comprises balancing about a 10-fold cross validation in a training set.

52. The method of claim 46, wherein the transcriptomic based biomarker-I (TBB-I) comprises markers selected from the group consisting of: 1553145_at (hypothetical protein FLJ39653), 1553575_at, 1557236_at (apolipoprotein L, 6), 1558142_at (trinucleotide repeat containing 6B), 1560752_at (F-box and WD-40 domain protein 2), 1565614_at (Zinc finger protein 337), 1567100_at (Dachshund homolog 1 (Drosophila)), 200068_s_at (calnexin///calnexin), 201031_s_at (heterogeneous nuclear ribonucleoprotein H1 (H)), 202646_s_at (cold shock domain containing E1, RNA-binding), 205758_at (CD8a molecule///CD8a molecule), 206188_at (zinc finger protein 623), 212637_s_at (WW domain containing E3 ubiquitin protein ligase 1), 212920_at, 213317_at (chloride intracellular channel 5), 213619_at (Heterogeneous nuclear ribonucleoprotein H1 (H)), 215443_at (thyroid stimulating hormone receptor), 216198_at (activating transcription factor 7 interacting protein), 217870_s_at (cytidylate kinase), 218087_s_at (sorbin and SH3 domain containing 1), 222145_at (CDNA: FLJ23572 fis, clone LNG12403), 223577_x_at (PRO1073 protein), 224321_at (transmembrane protein with EGF-like and two follistatin-like domains 2), 224373_s_at (IQ motif and WD repeats 1), 224644_at (CDNA clone IMAGE: 5278517), 226173_at (ornithine aminotransferase-like 1), 226773_at (CDNA FLJ35131 fis, clone PLACE6008824), 226880_at (nuclear casein kinase and cyclin-dependent kinase substrate 1), 228980_at (ring finger and FYVE-like domain containing 1), 229569_at (CDNA clone IMAGE:5263455), 231735_s_at (PRO1073 protein), 233765_at (Hypothetical LOC197135), 235803_at (Cytokine receptor-like factor 3), 236131_at (CDNA clone IMAGE:6622963), 236953_s_at (similar to RIKEN cDNA 8030451K01), 240544_at (Zinc finger, AN1-type domain 3), 240971_x_at (Cullin 4A), and 244042_x_at (Similar to retinoic acid receptor responder (tazarotene induced) 2).

53. The method of claim 46, wherein the transcriptomic based biomarker-II (TBB-II) comprises markers selected from the group consisting of: 1552419_s_at (tubulin tyrosine ligase-like family, member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (pre-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), and 43934_at (G protein-coupled receptor 137).

54. The method of claim 46, wherein a transcriptomic based biomarker-III (TBB-III) comprises markers selected from the group consisting of: 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE:5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 243766_s_at (TEA domain family member 2), and 244042_x_at.

55. A kit comprising a transcriptomic based biomarker-I (TBB-I): 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE: 5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 43766_s_at (TEA domain family member 2), 244042_x_at, complementary sequences, fragments, alleles, derivatives, variants and/or gene products thereof.

56. A kit comprising a transcriptomic based biomarker-II (TBB-II): 1552419_s_at (tubulin tyrosine ligase-like family; member 10), 1553212_at (keratin 78), 1555124_at (hypothetical protein MGC40574), 1556192_x_at (Metastasis suppressor 1), 1556320_at (Stomatin (EPB72)-like 1), 1556510_at (CDNA clone IMAGE:4796864), 1558484_s_at (leucine rich repeat containing 27), 1565662_at (Mucin 6, oligomeric mucus/gel-forming), 1567410_at (zinc finger protein 135), 1568513_x_at (Protease, serine, 1 (trypsin 1)), 1570408_at (Serine/threonine kinase 24 (STE20 homolog, yeast)), 203307_at (guanine nucleotide binding protein-like 1), 204581_at (CD22 molecule, myelin associated glycoprotein), 205586_x_at (VGF nerve growth factor inducible), 206333_at (musashi homolog 1 (Drosophila)), 207004_at (B-cell CLL/lymphoma 2), 210059_s_at (mitogen-activated protein kinase 13), 210228_at (colony stimulating factor 2 (granulocyte-macrophage)), 210384_at (protein arginine methyltransferase 2), 210923_at (solute carrier family 1 (glutamate transporter), member 7), 211024_s_at (thyroid transcription factor 1///thyroid transcription factor 1), 211062_s_at (carboxypeptidase Z///carboxypeptidase Z), 211096_at (we-B-cell leukemia transcription factor 2), 211181_x_at (runt-related transcription factor 1 (acute myeloid leukemia 1; aml1 oncogene)), 211710_x_at (ribosomal protein L4///ribosomal protein L4), 213096_at (transmembrane and coiled-coil domain family 2), 213121_at (small nuclear ribonucleoprotein 70 kDa polypeptide (RNP antigen)), 213242_x_at (KIAA0284), 213568_at (odd-skipped related 2 (Drosophila)), 213770_at kinase suppressor of ras 1 (214171_s_at (U2 small nuclear RNA auxiliary factor 2), 216116_at (NCK interacting protein with SH3 domain), 216427_at (CDNA: FLJ22786 fis, clone KAIA2150), 216820_at, 217054_at (CDNA FLJ39484 fis, clone PROST2014925), 217180_at (Hypothetical protein similar to KIAA0187 gene product), 217182_at (mucin 5AC, oligomeric mucus/gel-forming), 217322_x_at, 217430_x_at (collagen, type I, alpha 1), 219070_s_at (motile sperm domain containing 3), 219425_at (sulfotransferase family 4A, member 1), 221663_x_at (histamine receptor H3), 221684_s_at (Nyctalopin), 223974_at (hypothetical protein MGC11082), 226640_at (diacylglycerol lipase beta), 228074_at (hypothetical protein LOC162073), 229191_at (tubulin folding cofactor D), 229257_at (KIAA1856 protein), 229335_at (immunoglobulin superfamily, member 4C), 229358_at (Indian hedgehog homolog (Drosophila)), 230341_x_at (ADAM metallopeptidase with thrombospondin type 1 motif, 10), 230693_at (ATPase, Ca⁺⁺ transporting, cardiac muscle, fast twitch 1), 230768_at (FERM, RhoGEF and pleckstrin domain protein 2), 231510_at (GLI-Kruppel family member GLI2), 231629_x_at (Kallikrein-related peptidase 3), 231998_at, 233794_at (Single stranded DNA binding protein 3), 233974_s_at (family with sequence similarity 129, member B), 234495_at (kallikrein-related peptidase 15), 234637_at (keratin associated protein 4-5), 234881_at, 235568_at (chromosome 19 open reading frame 59), 235600_at (Transcribed locus), 236496_at (degenerative spermatocyte homolog 2, lipid desaturase (Drosophila)), 237087_at (Chromosome 14 open reading frame 105), 237144_at (Latent transforming growth factor beta binding protein 3), 237398_at (Transcribed locus), 237547_at (Hypothetical protein LOC728730), 237679_at (tripartite motif-containing 66), 238267_s_at, 238445_x_at (mannosyl (alpha-1,6-)-glycoprotein beta-1,6-N-acetyl-glucosaminyltransferase, isozyme B), 239026_x_at (centaurin, gamma 3), 239463_at (Transcribed locus), 239756_at (MAD1 mitotic arrest deficient-like 1 (yeast)), 240039_at (Transcribed locus///Transcribed locus), 240147_at (hypothetical protein MGC11257), 240517_at (Cystathionine-beta-synthase), 241270_at (Rhomboid 5 homolog 2 (Drosophila)), 241431_at, 242365_at (Coiled-coil domain containing 32), 243297_at (Vacuolar protein sorting 13 homolog D (S. cerevisiae)), 243497_at (Transcribed locus), 243766_s_at (TEA domain family member 2), 43934_at (G protein-coupled receptor 137), complementary sequences, fragments, alleles, derivatives, variants and/or gene products thereof.

57. A kit comprising a transcriptomic based biomarker-III (TBB-III): 1553212_at (keratin 78), 1557236_at (apolipoprotein L), 1558142_at (trinucleotide repeat containing 6B), 1558484_s_at (leucine rich repeat containing 27), 1565614_at (Zinc finger protein 337), 1565662_at (Mucin 6, oligomeric mucus/gel-forming), 1567100_at (Dachshund homolog 1 (Drosophila)), 203307_at (guanine nucleotide binding protein-like 1), 205758_at (CD8a molecule///CD8a molecule), 206333_at (musashi homolog 1 (Drosophila)), 212920_at, 213242_x_at (KIAA0284), 213619_at (Heterogeneous nuclear ribonucleoprotein H1 (H)), 213770_at (kinase suppressor of ras 1), 214171_s_at (U2 small nuclear RNA auxiliary factor 2), 215443_at (thyroid stimulating hormone receptor), 216198_at (activating transcription factor 7 interacting protein), 216427_at (CDNA: FLJ22786 fis, clone KAIA2150), 217054_at (CDNA FLJ39484 fis, clone PROST2014925), 217182_at (mucin 5AC, oligomeric mucus/gel-forming), 217322_x_at, 219425_at (sulfotransferase family 4A, member 1), 222145_at (CDNA: FLJ23572 fis, clone LNG12403), 229191_at (tubulin folding cofactor D), 229569_at (CDNA clone IMAGE:5263455), 231629_x_at (Kallikrein-related peptidase 3), 233765_at (Hypothetical LOC197135), 233794_at (Single stranded DNA binding protein 3), 233974_s_at (family with sequence similarity 129, member B), 234495_at (kallikrein-related peptidase 15), 235568_at (chromosome 19 open reading frame 59), 235803_at (Cytokine receptor-like factor 3), 236496_at (degenerative spermatocyte homolog 2, lipid desaturase (Drosophila)), 236953_s_at, 238445_x_at (mannosyl (alpha-1,6-)-glycoprotein beta-1,6-N-acetyl-glucosaminyltransferase, isozyme B), 239463_at (Transcribed locus), 240544_at (Zinc finger, AN1-type domain 3), 243766_s_at (TEA domain family member 2), 244042_x_at complementary sequences, fragments, alleles, derivatives, variants and/or gene products thereof.

58. A cell expressing any one or more biomolecules selected from TBB-I, TBB-II, or TBB-III.

59. A vector encoding any one or more biomolecules selected from TBB-I, TBB-II, or TBB-III.
